(19) United States
(12) Patent Application Publication
(10) Pub. No.: US 2024/0115672 A1
(43) Pub. Date: Apr. 11, 2024

(54) TREATMENT OF AN IOP CONDITION

(71) Applicant: THE RESEARCH FOUNDATION FOR THE STATE UNIVERSITY OF NEW YORK, Albany, NY (US)

(72) Inventor: Ioannis (John) DANIAS, Staten Island, NY (US)

(21) Appl. No.: 18/546,236

(22) PCT Filed: Feb. 11, 2022
(86) PCT No.: PCT/US2022/016043

§ 371 Date: Aug. 11, 2023

Related U.S. Application Data
(60) Provisional application No. 63/149,040, filed on Feb. 12, 2021.

(51) Int. Cl. A61K 38/49; A61K 9/00; A61P 27/06

(52) U.S. Cl. CPC A61K 38/49; A61K 9/0048; A61P 27/06; C12Y 304/21068

(57) ABSTRACT

Disclosed herein are methods of treatment for an intraocular pressure (IOP)-associated condition in a subject, that include administering to the subject an effective amount of a tissue plasminogen activator (tPA) therapeutic agent. In one embodiment, the IOP-associated condition is glaucoma. The administration of a tPA therapeutic agent can be an extended administration intended to cause a reduction in IOP in the subject for a period of at least one day to a year or more, relative to IOP levels in the subject prior to administration of the tPA therapeutic agent. The tPA therapeutic agent can be, for example, tPA, a tPA derivative, a small molecule direct or indirect tPA agonist, or a gene therapy vector.

### CROSS REFERENCE TO RELATED APPLICATIONS

This application claims the benefit of U.S. Provisional Patent Application No. 63/149,040, filed Feb. 12, 2021, the entire contents of which are incorporated by reference herein.

### STATEMENT REGARDING FEDERALLY SPONSORED RESEARCH OR DEVELOPMENT

This invention was made with government support under Contract No. NEI EY020670 and/or Contract No. R01 EY025543 awarded by the National Institutes of Health/National Eye Institute. The government has certain rights in the invention.

### BACKGROUND OF THE DISCLOSURE

The fibrinolytic system is a complex system of proteins that controls clotting of blood and subsequent dissolution of the resulting thrombus. Tissue plasminogen activator (tPA) and urokinase-type plasminogen activator (uPA, also known simply as urokinase) are two serine kinases that activate plasminogen by proteolytic cleavage. Activated plasminogen becomes plasmin with the ability to degrade fibrin. tPA also has activity at the cellular level for controlling extracellular matrix (ECM) remodeling and has been implicated in cell proliferation and migration.

tPA is a serine protease that is better known for its actions in regulating the fibrinolytic pathway. It lies upstream and can activate plasminogen into plasmin, which then degrades fibrin to dissolve blood clots. Yet, tPA has other roles in tissue homeostasis, either through plasmin or independent of it. tPA has, in addition to its enzymatic activity (that can directly or indirectly affect ECM components), non-enzymatic domains within its protein structure that can bind to distinct receptors eliciting a specific cellular response.

tPA activation is controlled by endogenous inhibitors, plasminogen activator inhibitors 1 (PAI1) and 2 (PAI2). PAI1 has been reported to be elevated in glaucoma in the past (Dan, J., et al., Arch Ophthalmol 123(2):220-4 (2005)) and has been shown to be synthesized by both the ciliary epithelium (Meyer, M. W., et al, Graefes Arch Gin Exp Ophthalmol 240(8):679-86 (2002)) and trabecular meshwork (TM) cells in response to TGF-β (Fuchshofer, R., et al., Exp Eye Res 77(6):757-65 (2003), Fleenor, D. L., et al., Invest Ophthalmol Vis Sci 47(1):226-34 (2006)), a known factor that induces reduction in outflow facility. tPA has also been reported to be down regulated in organ cultures after treatment with steroids (Snyder, R. W., et al., Exp Eye Res 57(4):461-8 (1993), Seftor, R. E., et al., J Glaucoma 3(4):323-8 (1994)). It appears that some of the effects of PAI are mediated through activation of matrix metalloproteinases (MMPs) in the TM (Fuchshofer, R., et al., Exp Eye Res 77(6):757-65 (2003)).

Steroid-induced intraocular pressure (IOP) elevation is caused by a decrease in aqueous humor outflow facility. Although the exact mechanism remains unclear, increased ECM deposition in the TM has been reported and is consistently detected in both glaucomatous human specimens and animal models of the disease.

The ECM structure of the conventional outflow pathway is dynamic and continually remodeled by matrix metalloproteinases (MMPs). MMPs are zinc endopeptidases that are secreted in their zymogen (pro-MMP) form for subsequent activation via proteolytic cleavage. The expression and activity of MMP-2, MMP-9 and MMP-13 are reduced in cases of primary open angle glaucoma and in animal models of ocular hypertension. Conversely, administration of exogenous MMPs, during anterior segment organ culture perfusion experiments, increases outflow facility.

MMP regulation occurs via cytokine-dependent transcriptional control and via proteolytic post-translational activation. tPA plays a critical role in fine-tuning both pathways of MMP activity regulation. tPA is expressed and secreted by TM cells under physiologic conditions. The proteolytic action of tPA allows it to activate pro-MMPs either through plasmin activation or through direct cleavage.

tPA also functions as a cytokine by promoting intracellular signaling cascades and gene expression changes following interactions with cell surface receptors, such as low-density-lipoprotein receptor-related protein 1 (LRP-1) and N-methyl-D-aspartate receptor (NMDAR). By this mechanism, tPA enhances MMP transcriptional expression in brain, retinal, lung and renal tissues.

The proteolytic action of tPA is dependent on the presence of serine-478 at its catalytic active site. Change of serine-478 to an alanine (S478A) results in complete loss of tPA enzymatic activity without affecting its binding properties to receptors and inhibitors allowing it to continue to function in a non-enzymatic fashion.

Previous studies have found that steroids cause a reduction in tPA at the TM and that exogenous administration of tPA can prevent and reduce steroid-induced IOP elevation in sheep and prevent steroid-induced reduction of outflow facility in mice. On the other hand, deletion of the gene encoding tPA (Plat) in mice causes a significant reduction in outflow facility. This effect is associated with a reduction in Mmp-9 expression in angle ring tissues of tPA deficient mice. Furthermore, over-expression of tPA in steroid treated mice results in increased expression of Mmp-2, Mmp-9 and Mmp-13 in angle ring tissue.

Recombinant human tPA (rh-tPA or h-tPA) has been used for the acute management of excessive fibrin in the anterior segment of the eye, and for the dissolution of subretinal hemorrhages. Common off-label use of tPA is for treatment of acute fibrin build-up in the immediate post-operative period following glaucoma surgery. Although short-term IOP reductions have been mentioned following treatment with tPA, they have been attributed to the dissolution of the fibrin clot in the anterior chamber. Accordingly, longer-term treatment with tPA, or administration of tPA in the absence of fibrin build-up, has not been suggested in the prior art. Long-term tPA administration is not recommended in the art for several reasons, for example, to avoid a risk of excessive ocular bleeding/hemorrhage.

Further, defects in the myocilin (MYOC) gene are the first described and most common genetic cause linked to glaucoma. They also account for approximately 8% of human juvenile glaucoma and are associated with high IOPs. While the function of the wildtype myocilin protein is unknown, mutant myocilin accumulates within the endoplasmic reticulum (ER) of TM cells, leading to their malfunction and subsequent apoptosis. The final effect is a reduction in aqueous humor outflow facility and increase in IOP. The Tyr437His (Y437H) mutation in exon 3 of MYOC is associated with one of the most severe disease phenotypes.

A transgenic mouse model having the human MYOC gene modified to contain the Y437H mutation (Tg-MYOCY437H) displays several glaucoma phenotype characteristics including IOP elevation and glaucomatous neurodegeneration. Alleviation of ER stress in these animals significantly improves aqueous humor outflow.

To date no attempt has been made to use tPA for therapeutic purposes in steroid induced glaucoma or other forms of glaucoma.

### BRIEF SUMMARY OF THE DISCLOSURE

Disclosed herein are methods of treatment for an intraocular pressure (IOP)-associated condition in a subject, that include administering to the subject an effective amount of a tissue plasminogen activator (IPA) therapeutic agent. In one embodiment, the IOP-associated condition is glaucoma. In one embodiment, an effective amount is an amount in the range of 10-200 μl.

In contrast to prior art uses of tPA for short-term or acute treatment, tPA therapeutic agents are disclosed herein as beneficial under conditions of extended or recurrent administration. The recurrent administration of the tPA therapeutic agent over an extended period of time, such as at least two weeks, at least one month, at least six months, or a year or more, can cause a reduction in IOP in the subject for a period of at least two weeks to a year or more, relative to IOP levels in said subject prior to administration of the tPA therapeutic agent.

The tPA therapeutic agent can be, for example, tPA; a tPA variant, an enzymatically inactive tPA variant, functional derivative, or homolog; a small molecule tPA agonist; an RNA molecule that causes tPA upregulation; a polypeptide or other molecule that causes tPA upregulation; an RNA molecule or other agent that down-regulates a negative regulator of tPA expression or activity; and a gene therapy vector. The tPA gene therapy vector can be a lentivirus or adeno-associated virus (AAV)-based vector or a non-viral vector encoding a tPA gene or a tPA derivative gene. The tPA gene therapy vector can contain a nucleic acid sequence encoding tPA; encoding a tPA functional derivative or homolog; encoding a polypeptide or other molecule that causes up-regulation of tPA expression; or encoding a polypeptide or other molecule that causes down-regulation of a negative regulator of tPA expression or activity.

In one embodiment, the tPA therapeutic agent is a small molecule tPA agonist, or an analog of a small molecule tPA agonist, the small molecule tPA agonist being selected from the group consisting of: an oxysterol, N-acetyl-cysteine, Neovastat, nicotine, allopregnanolone, testosterone, forskolin, L-threo-DOPS, PACAP, a PDE4 activator, 5-azacytidine, CPT-cAMP, retinoic acid, a phorbol ester, 8-bromo-cAMP, 2-diocynoyl-sn-glycerol (diC8), Phorbol 12 myristate 13 acetate (PMA), AF12198, CE3F4, Prostaglandin E2 (PGE2), Butyrate, 1,25-dihydroxyvitamin D-3, estradiol, an estrogen analogue, laminin, Interleukin-6 (IL-6), ascorbic acid, sesamol, and lysophosphatidylcholine. The tPA therapeutic agent can be administered by various methods, such as by intraocular injection.

The tPA therapeutic agent can be administered, for example, topically, systemically, by injection, by iontophoresis, or by implantation of cells that produce said tPA therapeutic agent.

### BRIEF DESCRIPTION OF THE FIGURES

FIGS. 1A-1B. Gene therapy with Adenovirus vectors carrying transgene (AdPLAT) can prevent or reverse steroid-induced reduced outflow facility. (A), Effect of AdPLAT on outflow facility in mice treated concurrently with the steroid triamcinolone acetonide (TA). TA, average outflow facility (μl/min/mmHg) of eyes treated with triamcinolone acetonide (TA) only. TA+PLAT, average outflow facility of eyes treated with TA and transfected with AdPLAT vector, showing expression of mCherry/AdPLAT. TA+/−PLAT, average outflow facility of eyes treated with TA and transfected with AdPLAT vector, showing no or minimal expression of mCherry/AdPLAT. TA+AdNull, average outflow facility of eyes treated with TA and transfected with control vector. (B), Effect of AdPLAT on outflow facility in mice pretreated with TA. TA. average outflow facility (μl/min/mmHg) of eyes treated with triamcinolone acetonide (TA) only. TA+AdPLAT, average outflow facility of eyes treated with TA and transfected with AdPLAT vector, showing expression of mCherry/AdPLAT. TA+/−AdPLAT, average outflow facility of eyes treated with TA and transfected with AdPLAT vector, showing no or minimal expression of mCherry/AdPLAT.

FIGS. 2A-2C. Device and method to measure outflow facility. (A), schematic for device used to measure outflow facility. (B), outflow facility measuring device, which includes a three-way valve which is connected to (i) a cannula for insertion into the eye; (ii) a flow-through pressure transducer; and (iii) a fluid reservoir. The pressure transducer is connected to a syringe loaded into a microdialysis infusion pump. For continuous pressure recording, the pressure transducer is attached to a bridge amplifier and the signal is fed into a chart or digital recorder. (C), eye is cannulated with a custom-made 33-gauge needle and connected via short PE60 tubing to the three-way valve.

FIG. 3. Effect of SCT (simvastatin, curcumin and troglitazone) mixture on outflow facility (μl/min/mmHg) after 1 week of TA injection. SCT administration improves outflow facility in treated eye of mice.

FIGS. 4A-4C. (A), Recombinant human tPA given as intravitreal injection can reverse IOP increase in sheep caused by steroid exposure. Steroids were administered to both eyes starting on Day 0 and tPA treatment commenced on Day 7. IOP (OD), IOP in control (left) eye treated with prednisolone only. IOP (OS), IOP in eye treated with prednisolone plus intravitreal tPA (right eye). Values are means+/−SEMs from 8 sheep. (B), The effect is not caused by the arginine present in the commercial tPA prep, as arginine administration alone does not alter steroid-increased IOP. (C). Administration of tPA prior to onset prevents IOP elevation. Representative experiment with 0.1 mg tPA injected into right eye (OD) and 4.23 mg arginine injected into left eye (OS) on Day 1; bilateral prednisolone instillations were also initiated on Day 1 and maintained through the end of protocol. tPA administration concurrent with steroid instillation kept outflow facility at normal levels.

FIG. 5. Outflow facility in PLAT KO (N=10), heterozygote (N=4) and wild type mice (N=8). Difference between WT and KO animals is significant (ANOVA, p<0.05, Tukey post hoc analysis).

FIGS. 6A-6D. Normalized fold change (mean±SD) (panels A and B) and fold difference (panels C and D) in expression of PAI-1. MMP-2, MMP-9, MMP-13, in mouse angle rings receiving triamcinolone (TA) alone or TA with an adenovector carrying sheep PLAT. TA+/+AdPLAT: eyes with significant PLAT expression in the TM, TA+/−AdPLAT: eyes without significant PLAT expression in the TM Fold changes were compared to 1. Asterisks indicate statistically significant differences (**p<0.01, ***p<0.001, t-test).

FIG. 7. Effect of tPA on gene expression in sheep. tPA acts through upregulation of specific MMPs.

FIG. 8. Relative fold difference of PLAT mRNA from 18S mRNA amounts in HTM cells treated with prednisolone acetate (PA treated) or vehicle (Control) for 1 hour. Differences arc statistically significant (p<0.05, t-test).

FIG. 9. Luciferase assay of PLAT promoter activity. A commercially available clone of the human PLAT promoter (Switchgear genomics) was used to transfect HTM cells using the nucleofector. Renila luciferase activity was measured 24 h after transfection in vehicle (Control) and prednisolone (PA) treated cells and normalized for cypridina activity in the medium.

FIG. 10A-10C illustrate various animal experimental setups and methods of treatment of samples. FIG. 10D is a graphical illustration of tPA activity of the tPA variants used in these experiments.

FIGS. 11A and 11B are images of treated eyes. FIG. 11C is an illustration of PLAT expression based on various treatments.

FIGS. 12A-12D are illustrations of outflow facility impact based on various treatments and effects on MMP expression.

FIGS. 13A-13D are illustrations of outflow facility impact based on various treatments and effects on MMP expression.

FIGS. 14A-14D are illustrations of outflow facility impact based on various treatments and effects on MMP expression.

FIGS. 15A-15E are illustrations of outflow facility impact based on various treatments and effects on MMP expression.

FIGS. 16A and 16B are illustrations of outflow facility impact based on various treatments.

FIGS. 17A-17D illustrate outflow facility impact based on various treatments for differing MMP expression.

FIGS. 18A-18D illustrate outflow facility impact based on various treatments and effects on MMP expression.

FIGS. 19A-19D are graphs that illustrate: (FIG. 19A) Outflow facility in Tg-MYOCY437H and wildtype littermate mouse eyes. Outflow facility (mean±SD μl/min/mmHg) was significantly reduced in Tg-MYOCY437H eyes (n=10) compared with wildtype (WT) littermate eyes (n=6) (****p<0.0001, T-test). Gene expression changes in Plat (FIG. 19B), Plau (FIG. 19C), and Pai-1 (FIG. 19D) were normalized (mean±SD) to values in WT littermate eyes. Expression was not significantly different between Tg-MYOCY437H angle ring tissues (n=11) and WT angle ring tissues (n=9) (p>0.05, ANOVA).

FIGS. 20A-20C are graphs that illustrate Mmp gene expression changes in Tg-MYOCY437H angle ring tissue. Gene expression changes in Mmp-2 (FIG. 20A), Mmp-9 (FIG. 20B), and Mmp-13 (FIG. 20C) were normalized (mean±SD) to values in wildtype (WT) littermate eyes. Group means were significantly different in Tg-MYOCY437H angle ring tissues (n=11) and WT angle ring tissues (n=9) for Mmp-2 expression and Mmp-9 expression (p<0.05 and p<0.05, T-test). Group means were not significantly different for Mmp-13 expression (p>0.05. T-test).

FIG. 21 is a graph that illustrates Tg-MYOCY437H mice do not have steroid-induced outflow facility reduction. Outflow facility (mean±SD μl/min/mmHg) was not further reduced in TA treated Tg-MYOCY437H eyes (n=7) compared with PBS treated Tg-MYOCY437H eyes (n=10) (p>0.05, T-test).

FIGS. 22A-22D are graphs that illustrate (FIG. 22A) Outflow facility in protein treated Tg-MYOCY437H mouse eyes. Outflow facility (mean±SD μl/min/mmHg) was significantly increased in eyes treated with tPA (n=10) and NEtPA (n=8) compared with those treated with BSA (n=17) (****p<0.0001, ANOVA with Tukey-Kramer post hoc analysis). The outflow facility of naïve (not treated with protein) wildtype (WT) littermate animals (n=6) are included for comparison purposes. The outflow facility in these eyes is different from the outflow facility in BSA, tPA and NE-tPA treated eyes. Gene expression changes in Mmp-2 (FIG. 22B), Mmp-9 (FIG. 22C), and Mmp-13 (FIG. 22D) were normalized (mean±SD) to values in WT littermate eyes. Group means were significantly different for Mmp-2, Mmp-9 and Mmp-13 expression in WT eyes (n=9), BSA eyes (n=16), tPA eyes (n=8) and NE-tPA eyes (n=8) (ANOVA, Mmp-2, p<0.0001, Mmp-9, p<0.01, Mmp-13, p<0.0001). Asterisks indicate differences on Tukey-Kramer post hoc analysis, *p<0.05, **p<0.01, ***p<0.001 ****p<0.0001.

FIGS. 23A-23D are graphs that illustrate (FIG. 23A) Outflow facility in protein and PBA treated Tg-MYOCY437H mouse eyes. Outflow facility (mean±SD μl/min/mmHg) was significantly increased in eyes treated with tPA (n=13) and PBA (n=14) compared with those treated with BSA (n=15) (****p<0.0001, ANOVA with Tukey-Kramer post hoc analysis). The outflow facility of naïve (not treated with protein or PBA) wildtype (WT) animals (n=6) are included for comparison purposes. The outflow facility in these eyes is different from the outflow facility in BSA and tPA treated eyes. Gene expression changes in Mmp-2 (FIG. 23B), Mmp-9 (FIG. 23C), and Mmp-13 (FIG. 23D) were normalized (mean±SD) to values in WT littermate eyes. Group means were significantly different for Mmp-2, Mmp-9 and Mmp-13 expression in WT eyes (n=9), BSA eyes (n=8), tPA eyes (n=8) and PBA eyes (n=8) (ANOVA, Mmp-2. p<0.0001, Mmp-9, p<0.001, Mmp-13, p<0.0001). Asterisks indicate differences on Tukey-Kramer post hoc analysis, *p<0.05, **p<0.01, ***p<0.001 ****p<0.0001.

### DETAILED DESCRIPTION OF THE DISCLOSURE

The present disclosure presents direct administration, activation or upregulation of tissue plasminogen activator (tPA) and tPA variants as a useful method for the treatment of intraocular pressure (IOP)-associated conditions, such as glaucoma.

The inventors have discovered that tPA therapeutic agents can be used to lower IOP long-term even when there is no obvious fibrin accumulation. This is in contrast to short-term use of tPA as indicated for the purpose of reducing excess fibrin in acute settings, such as following glaucoma surgery. The inventors have determined that administration of tPA therapeutic agents over an extended period of time can lower IOP and treat glaucoma and other conditions associated with increased IOP.

Intraocular pressure (IOP), the fluid pressure within the eye, can be measured in units of millimeters of mercury (mmHg) or kilopascals (kPa). Normal intraocular pressure is typically considered to be between 10 mmHg and 20 mmHg. The average value of intraocular pressure is 15.5 mmHg with fluctuations of about 2.75-3.50 mmHg. Elevated intraocular pressure (above 21 mmHg or 2.8 kPa) is the most important and only modifiable risk factor for glaucoma.

As used herein, the term “IOP-associated conditions” refers to conditions of the eye that are associated with elevated intraocular pressure. Examples of IOP-associated conditions include, but are not limited to, ocular hypertension and glaucoma, including primary glaucomas such as closed angle glaucoma and open angle glaucoma. Other forms of glaucoma, such as the various forms of developmental glaucomas and secondary glaucomas such as steroid-induced glaucoma, pigmentary glaucoma and pseudoexfoliation glaucoma, are also IOP-associated conditions according to the invention. Chronic forms of IOP-associated conditions, such as chronic forms of glaucoma or chronic ocular hypertension, in which the condition and/or the elevated IOP persist for extended periods of time (i.e., a persistent IOP-associated condition or elevated IOP lasting at least 1 day to 4 weeks, 1 to 12 months, or a year or more), can be effectively managed by the disclosed invention. In a preferred embodiment, the IOP-associated condition is glaucoma, particularly steroid-induced glaucoma and/or open angle glaucoma.

The “tPA therapeutic agent” or a “composition of the invention” can include one or more of: tPA; tPA functional derivates or homologs; tPA variants, such as recombinant IPA, particularly recombinant human tPA; small molecule tPA agonists; RNA and other molecules that cause tPA up-regulation; and gene therapy vectors.

tPA. tPA, or enzymatically active tPA, is an enzyme involved in the breakdown of blood clots. tPA catalyzes the conversion of plasminogen to plasmin, the major enzyme responsible for clot breakdown. There are at least three splice variants of tPA. As an example, the sequence for a specific human tPA variant, isoform 1, is set forth in GenBank Accession No. NM_000930.3. In a second example, the sequence for another specific human tPA variants, isoform 3, is set forth in GenBank Accession No. NM_033011. Transcript variant 3 is 46 amino acids shorter than variant 1 as it lacks exon 4 as present in transcript variant 1. The absence of these amino acids in transcript 3 makes it unlikely this isoform can form a two chain disulfide linked protein, like isoform 1. tPA polypeptide variants can be naturally-occurring, recombinant, modified, or synthetic and can include derivatives, analogs, and fragments of the tPA variant amino acid sequence. In addition, tPA genetic variants include naturally-occurring, recombinant, modified, or synthetic nucleic acids encoding tPA polypeptide variants and include derivatives, analogs, and fragments of a tPA gene variant or isoform.

Non-enzymatic, or enzymatically inactive, tPA (including variants) is used herein to denote all forms of tPA that are proteolytically inactive or have significantly reduced proteolytic activity (i.e. 50%, or less, 60%, 70%, 80%, 85%, 90% or 95%, or more, reduction in proteolytic activity relative to a negative control). The term non-enzymatic, or enzymatically inactive, tPA (including variants) furthermore encompasses all structural conformations of tPA provided that these structural conformations of tPA are proteolytically inactive or have significantly reduced proteolytic activity. The term non-enzymatic, or enzymatically inactive, tPA (including variants) furthermore encompasses tPA from all species including human and other relevant species, including mammals, such as primates, laboratory animals such as mice, and rats, and farm animals such as pigs and sheep.

Some non-limiting examples of enzymatically inactive tPA (including variants) include the following: Non-enzymatic, or enzymatically inactive, tPA (including variants) can be tPA or a variant thereof such that an active site serine can be mutated to alanine, rendering the enzyme catalytically inactive. Reduction or removal of catalytic activity of the non-enzymatic, or enzymatically inactive, tPA (including variants) can be due to block base deletions and/or one or more single base changes. One example of these mutations is at site S481A on the mature protein and S510A on the complete mRNA sequence (UniProtKB: locus TPA_MOUSE, accession P11214). The tPA still retains exosite binding as well as other biological properties of tPA including but not limited to surface binding to fibrin. In other examples, enzymatically inactive tPA can include variants with significant deletions of part of the catalytic domain of the tPA molecule. One possible example is a tPA variant that is missing a number (up to 130 or more) of amino acids from its carboxy terminal end. Such a deletion can render the catalytic domain inactive while not affecting the cytokine functionality of tPA and its ability to bind with specific receptors. Alternatively, one or more of the other protein domains that do not play a role in regulating outflow facility can be deleted while the catalytic domain is mutated to render the molecule enzymatically inactive as an enzymatically inactive, tPA (including variants thereof). Alternatively/in addition to the methods of inactivation above, enzymatically inactive tPA (including variants) can be forms of tPA that are produced by manipulation of the gene using site-directed mutagenesis or by truncation of the gene using restriction enzyme cleavage. One form is the truncated protein constructed by deletion of codons encoding Leu₄₁₁-Pro₅₂₇ by cleavage of the cDNA at an Sst1/SacI site. Other mutations comprise deletion of or replacement of the serine residue at position 478, with a conservative amino acid substitution such as by alanine, glycine, or threonine. Other enzymatic activity-destroying mutations in the serine protease region are also included in the scope of the disclosure. Alternatively/in addition to the methods of inactivation above, enzymatically inactive tPA (including variants) can be, native tPA that was inactivated by isolation of the active site using the peptide inhibitor PPACK (D-phenylalanyl-L-prolyl-L-arginine chloromethylketone). tPA functional derivatives. Functional derivatives and homologs of tPA are contemplated for use in the disclosed methods. As used herein, a “functional derivative” is a molecule which possesses the capacity to perform the biological function of tPA, i.e, a molecule that is able to functionally substitute for tPA, e.g., in the ability to catalyze the conversion of plasminogen to plasmin, improve outflow facility, and/or reduce elevated IOP. Functional derivatives include fragments, parts, portions, equivalents, analogs, mutants, mimetics from natural, synthetic or recombinant sources including fusion proteins. Derivatives may be derived from insertion, deletion or substitution of amino acids. Amino acid insertional derivatives include amino and/or carboxylic terminal fusions as well as intrasequence insertions of single or multiple amino acids. Insertional amino acid sequence variants are those in which one or more amino acid residues are introduced into a predetermined site in the polypeptide although random insertion is also possible with suitable screening of the resulting product. Deletional variants are characterized by the removal of one or more amino acids from the sequence. Substitutional amino acid variants are those in which at least one residue in the sequence has been removed and a different residue inserted in its place. Additions to amino acid sequences include fusions with other peptides, polypeptides or proteins.

A “homolog” is a protein related to a second protein by descent from a common ancestral DNA sequence. A member of the same protein family (for example, the tPA family) can be a homolog. A “functional homolog” is a related protein or fragment thereof that is capable of performing the biological activity of the desired gene, i.e, is able to functionally substitute for tPA. Homologs and functional homologs contemplated herein include, but are not limited to, polypeptides derived from different species.

A functional derivative or homolog can have 75%, 80%, 85%, 90%, 95% or greater amino acid sequence identity to a known tPA amino acid sequence, or 75%, 80%, 85%, 90%, 95% or greater amino acid sequence identity to a tPA variant thereof.

tPA variants and homologs. A “variant” refers to a molecule substantially similar in structure and function to either the entire molecule, or to a fragment thereof. Thus, as the term variant is used herein, two molecules are variants of one another if they possess a similar activity even if the structure of one of the molecules is not found in the other, or if the sequence of amino acid residues is not identical. The term variant includes, for example, splice variants or isoforms of a gene. Equivalents should be understood to include reference to molecules which can act as a functional analog or agonist. Equivalents may not necessarily be derived from the subject molecule but may share certain conformational similarities. Equivalents also include peptide mimics.

tPA variants include recombinant tissue plasminogen activators (r-tPAs) such as alteplase, reteplase, tenecteplase, and desmoteplase. Reteplase is a recombinant non-glycosylated form of htPA modified to contain 357 of the 527 amino acids. Tenecteplase is a recombinant fibrin-specific plasminogen activator derived from native t-PA by modifications at three sites of the protein structure. Both reteplase and tenecteplase are FDA approved. Other investigational molecules exist with similar activity, some of them modified (2^{nd} and 3^{rd} generation) rtPAs, some are molecules that share similarities with tPA but come from other organisms, for example, anistreplase, duteplase, monteplase, lanoteplase, pamiteplase, amediplase, desmoteplase, staphylokinase, snake venom plasminogen activators such as TSV-PA (Trimeresurus stejnegeri venom plasminogen activator), Haly-PA (Agkistrodon halys venom plasminogen activator), LV-PA (Lachesis muta muta venom plasminogen activator), and recombinant chimeric CPAs such as GHRP-SYQ-K2S (which includes the IPA kringle 2 domain, K2S, and the tPA serine protease domain, glycyl-histidyl-arginyl-prolyl) and GHRP-scu-PA-32K (glycyl-histidyl-arginyl-prolyl-single-chain urokinase-type plasminogen activator). For review see Flemmig and Melzig (Flemmig, M., et al., J Pharm Pharmacol 64(8):1025-1039 (2012)), the contents of which are incorporated by reference herein.

A “tPA agonist” is a molecule that increases the expression, activity or function of tPA. For example, a compound can act as a tPA activator by increasing or enhancing tPA expression or activity, or increasing or enhancing the tPA-mediated catalysis of plasminogen to plasmin. Examples of tPA agonists include peptides, polypeptides, proteins, antibodies, small molecules, chemotherapeutic agents, and fragments, derivatives and analogs thereof, that increase or enhance the expression, activity or function of tPA.

Small molecule tPA agonists. A number of small molecules are known to be tPA agonists. including, but not limited to:
a. 20-S-Hydroxycholesterol and other oxysterols (Sonic-Hedghog activators) (Xin, H., et al., J Cereb Blood Flow Metab 31(11):2181-2188 (2011)), (Dwyer, J. R., et al., J Biol Chem 282(12):8959-8968 (2007))
b. N-acetyl-cysteine (Chu, D. I. et al., Surgery 149(6):801-812 (2011))
c. Neovastat (CAS Registry No. 305838-77-1) (Gingras, D., et al., Biochem Biophys Res Comma, 320(1):205-212 (2004))
d. Nicotine (Katono, T., et al., Arch Oral Biol 54(2):146-155 (2009))
e. Allopregnanolone (3α,5α-tetrahydroprogesterone) (VanLandingham, J. W., et al., J Cereb Blood Flow Metab 28(11):1786-1794 (2008))
f. Testosterone (Guo, J., et al., Endocrine 32(1):83-89 (2007))
g. Forskolin (Guo, J., et al., Endocrine 32(1):83-89 (2007))
h. L-threo-DOPS (L-threo-dihydroxyphenylserine) (Mataga, N., et al., Neurosci Lett 218(3):149-152(1996))
i. Pituitary adenylate cyclase-activating polypeptide (PACAP) (Raoult, E., et al., J Neurochem 119(5):920-931 (2011))
j. PDE4 activators (e.g., lloprost, CAS Registry No. 73873-87-7) (Yang, F., et al., Thromb Res 129(6):750-753 (2012))
k. 5-azacytidine (Griffiths, J. B., et al., Dev Biol Stand 66:417-422 (1987))
l. CPT-cAMP (CAS Number 129735-01-9) (Heaton, J. H., et al., Mol Endocrinol 3(1):185-192 (1989))
m. Retinoic acid (Benjamin, L. A., et al., Cancer Chemother Pharmacol 25(1):25-31 (1989))
n. Phorbol esters (Grulich-Henn, J., et al., Blut 61(1):38-44 (1990))
o. 8-bromo-cAMP (Heaton, J. H., et al., Mol Endocrinol 4(1):171-178 (1990))
p. 2-diocynoyl-sn-glycerol (diC8) (Grulich-Henn, J., et al., Blut 61(1):38-44 (1990))
q. Phorbol 12 myristate 13 acetate (PMA) (Grulich-Henn, J., et al., Blut 61(1):38-44 (1990))
r. Interleukin 1 antagonists (e.g., AF12198, CAS Registry No. 185413-30-3) (Bevilacqua, M. P., et al., J Clin Invest 78(2): 587-591 (1986))
s. Epac1 inhibitors (e.g., the tetrahydroquinoline analog CE3F4) (Courilleau. D., et al., J Biol Chem 287(53):44192-44202 (2012))
t. Prostaglandin E2 (PGE2) (Markosyan, N., et al., Endocrinology 150(1):435-444 (2009))
u. Butyrate (Reinders, J. H., et al., Ann N Y Acad Sci 667:194-198 (1992))
v. 1,25-dihydroxyvitamin D-3 (Fukumoto, S., et al., Biochim Biophys Acta 1201(2):223-228 (1994))
w. Estradiol and estrogen analogues (Davis, M. D., et al., J Steroid Biochem Mol Biol 52(5):421-430 (1995))
x. Laminin (Sonohara, S., et al., Int J Cancer 76(1):77-85 (1998))
y. Interleucin-6 (IL-6) (Hosoya, S., et al., J Enciod 24(5):331-334 (1998))
z. Ascorbic acid (Yoshino, A., et al., Life Sci 70(12):1461-1470 (2002))
aa. Sesamol (Chen, P. R., et al., J Nutr Biochem 16(1):59-64 (2005))
bb. Lysophosphatidylcholine (Sheikh, A. M., et al., Biochem Biophys Res Commun 329(1):71-77 (2005))

Many of these molecules have been shown to increase tPA in various cells in culture or in vivo.

It is a subject of this invention that tPA therapeutic agents of the invention preferably upregulate tPA expression, function, or activity in the trabecular meshwork (TM), an important area of the eye for regulating IOP. tPA activity in the TM would be particularly effective to reduce IOP.

RNA molecules that cause tPA up-regulation. In addition, a tPA therapeutic agent can be an RNA molecule that up-regulates tPA expression. Such molecules include antisense oligonucleotides, ribozymes, and/or short interfering RNA (siRNA) directed against genes that negatively regulate tPA, such that reduced expression of these negative regulators causes increased expression or activity of tPA. Negative regulators of tPA include the genes encoding for Tenascin C, Hypoxia Inducible Factor 1 (HIF1), Exchange Protein directly Activated by cAMP (EPAC1), interleukin 1 and Patched 1.

Gene therapy. Gene therapy vectors can be any vector that can effectively increase tPA expression in the eye, including vectors that encode an enzymatically inactive tPA variant. Many vectors useful for transferring exogenous genes into target mammalian cells are available. The vectors may be episomal, e.g. plasmids or virus derived vectors such cytomegalovirus vector, adenoviral vector, adeno-associated viral (AAV) vector, etc., or the vectors may be integrative, e.g., integrating the reprogramming gene into the target cell genome, through homologous recombination or random integration, e.g. retrovirus derived vectors such MMLV (Moloney Murine Leukemia Virus), HIV-1, ALV (Avian leukosis virus), or lentiviral vectors.

In one embodiment, a vector for expressing a tPA therapeutic gene comprises a promoter operably linked to the tPA therapeutic gene. The phrase “operably linked” or “under transcriptional control” as used herein means that the promoter is in the correct location and orientation in relation to a polynucleotide to control the initiation of transcription by RNA polymerase and expression of the polynucleotide. Several promoters are suitable for use in the vectors for expressing the reprogramming factor, including, but not limited to, RNA pol I promoter, RNA pol II promoter, RNA pol III promoter, and cytomegalovirus (CMV) promoter. Other useful promoters are discernible to one of ordinary skill in the art. In some embodiments, the promoter is an inducible promoter that allows one to control when the tPA therapeutic gene is expressed. Suitable examples of inducible promoters include tetracycline-regulated promoters (tet on or tet off) and steroid-regulated promoters derived from glucocorticoid or estrogen receptors. Constitutive expression of a tPA therapeutic gene can be achieved using, for example, expression vectors with a CMV, CAG (chicken beta-actin promoter with CMV enhancer), or PGK (phosphoglycerate kinase 1) promoter. Inducible expression of a tPA therapeutic gene can be achieved using, for example, a tetracycline responsive promoter, such as the TRE3GV (Tet-response element 3rd generation) inducible promoter (Clontech Laboratories, Mountain View, CA). Alternatively, the promoter operably linked to the tPA therapeutic gene may be a promoter that is activated in specific cell types and/or at particular points in development.

Depending on the promoter used, expression of a tPA therapeutic gene can be constitutive (continuous expression of the factor) or inducible (capable of being turned on and off). Expression can also be transient, that is, temporary expression of the tPA therapeutic gene over a limited time span. Transient expression may be achieved by use of a non-integrative vector, where the vector is lost from the cell or cell population over time, or by use of an inducible promoter in an integrative or non-integrative vector that can be manipulated to cease expression of the reprogramming gene after a period of time. In a specific embodiment, expression of a tPA therapeutic gene is inducible.

Suitable vectors can contain markers to identify and/or select transformed cells. Examples of selectable markers include visual markers such as green fluorescent protein (GFP), red fluorescent protein (RFP), or fluorescein; epitope markers such as His, c-myc, GST, Flag, or HA tags; enzymatic/nutritional markers such as DHFR (dihydrofolate reductase); or antibiotic resistance markers such as neomycin. puromycin, blasticidin, or hygromycin.

Preferred gene therapy vectors include AAV2 vectors, preferably self-complimentary AAV2 (scAAV2) vectors, and lentivirus vectors, encoding the tPA gene. AAV2 and lentivirus vectors can provide long term expression of proteins in the TM. The inventors have used lentiviral vectors to express proteins in the TM in animals. Expression can be maintained for at least a period of months and can last for years. ScAAV2 vectors have also been used for the long-term transfection of animals (up to 2 years) without adverse effects. Because of the size of the gene encoding the tPA native protein, scAAV vectors may not be able to accommodate the whole insert, requiring alternative strategies for delivery of the transgene. However, the message for modified tPA proteins that are smaller in size can be packaged in scAAV2. For gene therapy experiments it is important to incorporate controls that will allow the cessation of expression if needed (and potentially to also allow exogenous induction of expression). These controls can be encoded in the genetic material and can, for example, include Tetracycline- or tamoxifen-inducible repression systems.

Gene therapy encompasses expression of any tPA or tPA variant or derivative, as well as expression of a gene encoding a positive regulator of tPA, or encoding a naturally-occuring, recombinant, modified, or synthetic protein, variant or derivative or other molecule that up-regulates tPA, or encoding a negative regulator of a gene that negatively regulates tPA. Positive regulators of tPA include Sonic Hedgehog (Shh), protein kinase A, laminin and interleukin 6.

Genes for molecules that downregulate expression of tPA (“tPA inhibitors”) can be perturbed by antisense oligonucleotides, siRNA or ribozymes. Thus, antisense oligonucleotides, siRNA or ribozymes that reduce or prevent expression of inhibitors that downregulate tPA are also contemplated as tPA therapeutic agents. Genes that downregulate tPA include, but are not limited to:
a) Tenascin C (Brellier, F., et al., FEBS Lett 585(6):913-920 (2011)).
b) HIF1 (Zhu, G., et al., Osteoarthritis Cartilage 17(11):1494-1502 (2009)).
c) Epac1 (Yang, F., et al., Thromb Res 129(6):750-753 (2012)).
d) Interleukin 1 (Bevilacqua, M. P., et al., J Clin Invest 78(2): 587-591 (1986)).
e) Patched 1. Patched 1 is inhibited by sonic hedgehog (SHH); SHH upregulates tPA by inhibiting Patched 1 (Xin, H., et al, J Cereb Blood Flow Metab 31(11): 2181-2188 (2011)).

Methods of treatment. This disclosure presents methods for the treatment of IOP-associated conditions, by administering an effective amount of a tPA therapeutic agent to a subject in need thereof. The tPA therapeutic agent can be administered to the subject for as long as the condition and/or elevated IOP persists, in any manner that provides extended administration of the tPA therapeutic agent and/or long-term reduction of IOP. A “reduction” or “lowering” of IOP encompasses reduction of IOP to within normal levels of 10-20 mmHg, or any lowering of IOP, such as by 3, 5, 8, or 10 mmHg or more in a subject, relative to before treatment of said subject was commenced. “Long-term reduction” in IOP can be a reduction in IOP lasting 1 day to 4 weeks, 1 to 12 months, or a year or more. Extended administration includes, but is not limited to, less frequent administration of a composition that provides extended release or extended expression of a tPA therapeutic agent, or more frequent administration of a composition that provides shorter acting release or expression of a tPA therapeutic agent.

In the methods contemplated herein, the tPA therapeutic agents can be administered for at least 1, 2, 3, 4, 5, 6, 7, or 8 weeks, or for at least 1, 2, 3, 4, 5, 6, 7, 8, 9, 10, 11, or 12 months, or at least 1, 2, 3, 4, or 5 years. As part of the extended administration, the tPA therapeutic agent can be administered on a recurrent or repeated basis, such as on a daily, weekly, bi-weekly, monthly, bi-monthly, or on an annual basis, to provide a reduction in IOP over periods of time such as 1 day to 4 weeks, 1 to 12 months, or a year or more. This recurrent basis differs from prior art suggestion to administer tPA to treat acute fibrin build-up, such as administration of a single or limited number of intraocular tPA injections, in the immediate post-operative period following ocular surgery, to reverse fibrin accumulation. Prior to the instant invention, tPA administration in the absence of fibrin accumulation, or extended tPA treatment as a long-term solution to the problem of chronic elevated IOP, had not been suggested in the art. However, the disclosure herein encompasses both tPA administration even where excess fibrin accumulation is not apparent, and extended tPA treatment as a long-term solution to the problem of chronic elevated IOP.

Alternatives to arginine formulations: Although unmodified recombinant tPA is formulated with L-Arginine, other modified tPAs, such as reteplase, tenecteplase, and other tPA functional derivatives, as discussed above, do not use Arginine. Formulas encompassing IPA therapeutic agents, including tPA, IPA functional derivatives and variants, and small molecule tPA therapeutic agents, in a formulation with low or no Arginine, are preferred in the methods of the invention.

As used herein, the terms “subject” and “patient” are used interchangeably and refer to an animal, preferably a mammal such as a non-primate (e.g., cows, pigs, horses, cats, dogs, rats etc.) and a primate (e.g., monkey and human), and most preferably a human.

As used herein, “treatment” refers to clinical intervention in an attempt to alter the disease course of the individual or cell being treated. Therapeutic effects of treatment include without limitation, preventing recurrence of disease, alleviation of symptoms, diminishment of any direct or indirect pathological consequences of the disease, decreasing the rate of disease progression, amelioration or palliation of the disease state, and remission or improved prognosis. For example, treatment of a glaucoma patient can include lowering IOP and/or preventing, reducing, or ameliorating eye pain, optic nerve damage. retinal cell damage. or retinal cell loss. Although lowering IOP is preferably lowering IOP to within normal levels of 10-20 mmHg, any lowering of IOP, such as by 1-10 mmHg, 10-20 mmHg, or 20 mmHg or more in a subject, relative to before treatment was commenced, is considered to be effective.

The inventors have determined that administration of a tPA therapeutic agent can improve aqueous humor outflow in the eye. Aqueous humor is the clear, watery fluid that fills the complex space in the front of the eye which is bounded at the front by the cornea and at the rear by the front surface or face of the vitreous humor. Production, circulation, and drainage of aqueous humor into and out of the anterior chamber of the eye maintains the IOP at a relatively constant level.

The trabecular meshwork is the sponge-like tissue located near the cornea and iris that functions to drain the aqueous humor from the eye. The trabecular meshwork offers a certain resistance to the outflow of aqueous humor that is needed to maintain a steady-state IOP. The inverse of this resistance is trabecular outflow facility, a measure of the compliance of the trabecular meshwork. In glaucomatous eyes the resistance to aqueous humor outflow is increased due to an increase in different forms of extracellular material deposited within the meshwork, which decreases outflow facility. The inventors have found that tPA therapeutic agents can increase the outflow facility of the trabecular meshwork, which improves IOP and reduces the risk of, or directly treats, IOP-related conditions.

As used herein, the terms “therapeutically effective amount” and “effective amount” are used interchangeably to refer to an amount of a composition of the invention that is sufficient to result in the prevention of the development, recurrence, or onset of an TOP-associated condition and one or more symptoms thereof; enhance or improve the prophylactic effect(s) of another therapy; reduce the severity and duration of an IOP-associated condition; ameliorate one or more symptoms of an IOP-associated condition, in particular to lower IOP and/or improve outflow facility; prevent the advancement of an TOP-associated condition; cause regression of an IOP-associated condition; and/or enhance or improve the therapeutic effect(s) of additional treatment(s) administered to ameliorate an IOP-associated condition.

A therapeutically effective amount can be administered to a patient in one or more doses sufficient to palliate, ameliorate, stabilize, reverse or slow the progression of the IOP-associated condition, or otherwise reduce the pathological consequences of the condition, or reduce the symptoms of the condition. The amelioration or reduction need not be permanent, but may be for a period of time ranging from at least one hour, at least one day, or at least one week or more. The effective amount is generally determined by the physician on a case-by-case basis and is within the skill of one in the art. Several factors are typically taken into account when determining an appropriate dosage to achieve an effective amount. These factors include age, sex and weight of the patient, the condition being treated, the severity of the condition, as well as the route of administration, dosage form and regimen and the desired result.

Administration to a subject in need of treatment. The tPA therapeutic agent or composition of the invention can be administered to an eye of a patient as solutions, suspensions, or emulsions (dispersions). For example, the composition can be delivered topically to the eye in the form of drops, sprays, or gels. It can also be absorbed into contact lens or other non-biodegradable or biodegradable material that is placed on the cornea or conjunctiva. Alternatively, the composition can be administered by injection (e.g., intravitreal, intraorbital, subconjunctival, supraciliary and/or sub-tenon injection). The composition can also be administered by means of an implantable device, which can be attached, for example, to a subconjunctival, anterior chamber or vitreous region of the eye. For administration to a patient, the agent or composition is prepared with pharmaceutically acceptable opthalmologic carriers, excipients, or diluents.

Other ingredients which may be desirable to use in the ophthalmic preparations of the present invention include preservatives, co-solvents, buffers, viscosity building agents and penetration enhancers. Viscosity building agents, such as hydroxymethyl cellulose, hydroxyethyl cellulose, methylcellulose, polyvinylpyrrolidine, a polymer matrix such as CAPA4101 or the like, may be added to the compositions of the present invention to improve the retention of the compound in the conjunctival sac or surrounding area. In order to prepare sterile ophthalmic ointment formulations, the tPA therapeutic agent may be combined with a preservative in an appropriate vehicle, such as white petroleum, mineral oil or liquid lanolin. Sterile ophthalmic gel formulations may be prepared by suspending the IPA therapeutic agent in a hydrophilic base prepared from the combination of, for example, carbopol-940, or the like, according to the methods known in the art for other ophthalmic formulations.

Protein molecules like tPA or modified tPA functional derivatives are preferably administered intraocularly, as their penetration of the ocular wall is limited. Intracameral injections (injections into the anterior chamber) are easier to perform, but proteins injected there are cleared fairly rapidly. Proteins injected intravitreally are eliminated in large part (˜70%) through the anterior chamber. Because diffusion in the vitreous is delayed, proteins injected there have a longer duration of action. For this reason, intravitreal injection of tPA agents is preferred for intraocular injection. Formulations of tPA and tPA analogues and functional derivatives, for injection and administration by other routes, are known in the art.

The inventors have determined that, in a sheep model of elevated IOP, the effect of a single tPA injection on IOP lasted for approximately 18 days. Administration in subjects such as humans are expected to show a similar time frame for effect on IOP (i.e., two to three weeks). Recurrent (for example, monthly) intravitreal injections are widely accepted for therapy of other ocular conditions (like macular degeneration), but are more involved and carry a higher risk for infection. Therefore, although recurrent injections of a tPA therapeutic agent are encompassed by the invention, the skilled artisan or doctor must weigh the benefit of such repeat injections with the potential negative effects.

Other sites and modes of administration include topical administration, administration via iontophoresis; implantation of cells that are genetically engineered to constantly produce a tPA therapeutic agent; implantation of slow release device; and subconjunctival administration. A review of methods for administration of ophthalmic drugs is found in Kompella, et al., Ther Deliv 1:435-456 (2010), the contents of which are incorporated by reference.

Topical administration: Although tPA has been shown to penetrate the cornea, administration via this mode has the disadvantage of limited penetration. In addition a significant amount of the tPA therapeutic agent may be released in the tears, potentially causing side effects in the nasal cavity and upper respiratory system.

Administration via iontophoresis: a tPA therapeutic agent can be administered iontophoretically. Iontophoresis utilizes low currents to enhance the penetration of charged molecules across tissue barriers. The drug is applied using an electrode carrying the same charge as the drug. An electrode with the opposite charge placed elsewhere in the body completes the circuit. The ionized drug molecule penetrates the tissue by electric repulsion. Additionally, neutral molecules can potentially be delivered using iontophoresis on the basis of electro-osmosis or solute-associated fluid transport. Commercially available iontophoresis devices include OCUPHOR (Iomed Inc., USA) and VISULEX (Aciont Inc., USA). Such delivery method will avoid some of the problems of topical administration while minimizing effects on the posterior segment of the eye.

Implantation of cells that are genetically engineered to constantly produce a tPA therapeutic agent: cells that are genetically engineered to produce a tPA therapeutic agent can be implanted within the eye. Such engineered cells may, for example, reside within a permeable device that allows diffusion of their protein products in the eye (such as Encapsulated Cell Technology, available from Neurotech Pharmaceuticals). Such a device or cells can be implanted surgically in the posterior or anterior segment of the eye and provide for extended administration of specific doses of tPA.

Implantation of slow release devices: a tPA therapeutic agent formulated in a slowly biodegradable substrate, for example, poly(lactic-co-glycolic) acid PLGA) or polylactic acid (PLA), can be implanted in the anterior (or posterior) segments surgically and allowed to release tPA therapeutic agent over long periods of time (up to, or even more than, 2 years). The device can also reside outside the eye (in the subconjunctival space) and connect with the anterior chamber (AC) via a small tube. In such case discharge of the medication can be controlled externally, and the device can be refillable.

Subconjunctival administration (injection through the conjunctiva) is also contemplated herein.

Small molecules that stimulate increased expression of tPA can be delivered by all of the above methods as well as orally. In one embodiment, small molecules are delivered topically.

Dosage of tPA therapeutic agents. tPA has been used in acute situations by intracameral injection usually at a dosages of 10-25 μg (Kim, M. H., et al., Ophthalmic Surg Lasers 29(9):762-766 (1998), Wu, T. T., et al., Eye (Lond) 23(1):101-107 (2009)). For intravitreal use it has been used to dissolve sub-macular hemorrhages at a dosage of 30-100 μg (Chen, C. Y., et al., Retina 27(3):321-328 (2007)). Retinal toxicity has been reported with doses above 50-100 lag, but toxicity was attributed to the presence of arginine in the commercial preparations (Chen, S. N., et al., Ophthalmology 110(4):704-708 (2003), Oh, H. S., et al., Curr Eye Res 30(4):291-297 (2005)). For extended administration of tPA, and tPA variants and functional derivates such as reteplase, tenecteplase, and other modified tPAs, injections of 10-120 μg per treatment, preferably 30-100 μg per treatment, even more preferably 25-50 μg, can be administered to a subject in need of treatment. In some embodiments, tPA therapeutic agents as disclosed herein are administered at dosages of 10, 20, 25, 30, 35, 40, 45, 50, 55, 60, 65, 70, 75, 80, 85, 90, 95, 100, 110, or 120 μg per dose. The doctor or skilled artisan can modify these dosages appropriately.

This disclosure also encompasses very low dose administration in a slow-release formulation or device. The inventors have achieved a reduction in IOP with a single dose of 1 ng of tPA injected intracamerally. Turnover of aqueous is approximately 120 minutes, so the injected agent is completely gone by 120 minutes. Assuming a linear model of elimination, in 30 minutes there would be 75% (of 1 ng) present. Thus the low dosage formulations and devices disclosed herein would release approximately 1 ng or more every 30 minutes. Accordingly, the slow release device disclosed herein provides continuous release of 0.1-5 ng, preferably 1 ng, of tPA therapeutic agent every 30 minutes. That means release rate should be about 20-100 ng/day, preferably 40-80 ng/day, most preferably about 48-50 ng/day.

Throughout this application, the terms “about” and “approximately” indicate that a value includes the inherent variation of error for the device, the method being employed to determine the value, or the variation that exists among the study subjects. In one non-limiting embodiment the terms are defined to be within 10%, preferably within 5%, more preferably within 1%, and most preferably within 0.5%.

For example, based on continuous administration of 48-50 ng/day, a 6 month continuous delivery of tPA therapeutic agent would contain approximately 9000 ng (9 μg) of tPA therapeutic agent. Accordingly, a concentration of 9 μg tPA per 2 μl formulation, or about 5 μg/μl, or 5 about mg/ml, is placed, for example, inside a 1×1×2 mm device. Currently tPA therapeutic agents are administered as solution, but solid forms are contemplated for administration in a slow release device.

The present disclosure encompasses treating IOP-associated conditions, such as glaucoma, by upregulating tPA but also by downregulating plasminogen activator inhibitors 1 (PAI1) and 2 (PAI2). Thrombin is a known upregulator of tPA while metformin, and PPAR agonists (like troglitazone and rosiglitazone) are known antagonists of the PAIs. In addition statins are known to downregulate PAIs systemically and can be used either systemically or topically to modulate this system. Other activators of tPA have also been described, as well as other small molecules that inhibit PAIs and can be used for the same purposes. Statins also have direct effects on tPA activation (Essig, M., et al., Circ Res 83(7):683-90 (1998a), Essig, M., et al., J Am Soc Nephrol 9(8):1377-88 (1998b), Asahi, M., et al., J Cereb Blood Flow Metab 25(6): 722-9 (2005), Aarons, C. B., et al. Ann Surg 245(2):176-84 (2007)), in some instances without affecting PAIs.

Beginning at Example 10 below, experiments were done in order to determine whether tPA regulates aqueous humor outflow via its proteolytic and/or cytokine action at the level of the TM, native tPA and mutant non-enzymatically (NE) active tPA (NE-tPA/S478A-tPA) were used in a mouse model of steroid-induced glaucoma, in animals under baseline conditions, in PlatKO mice and in Mmp-9KO mice. The effect of tPA on outflow facility and Mmp expression was explored in these animals.

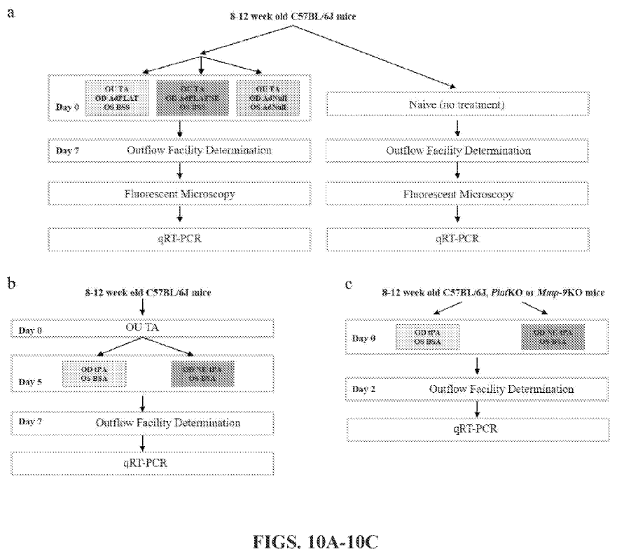

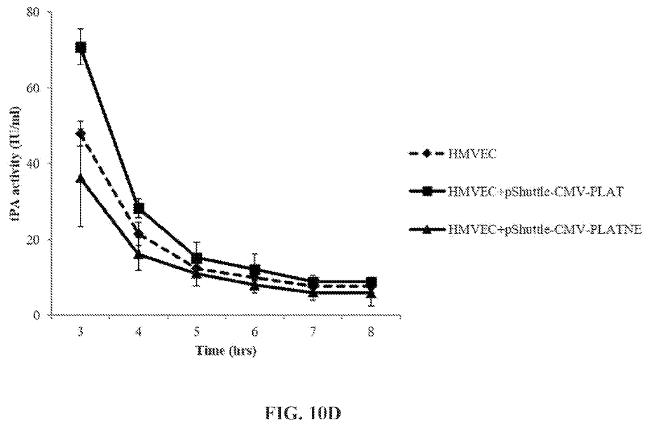

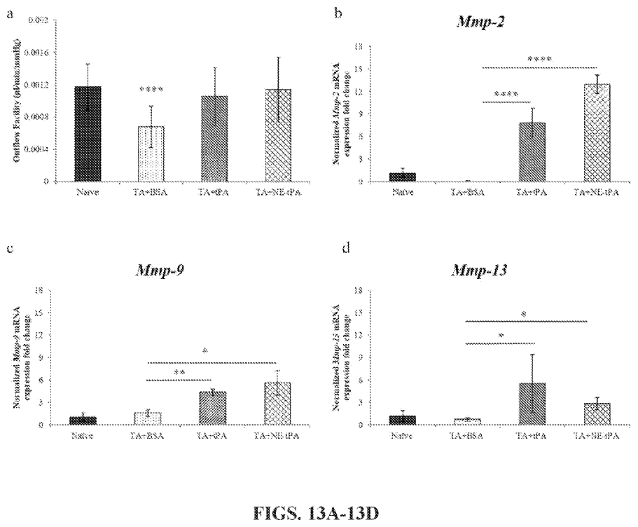

It has been reported that plasminogen levels were below detection limits in anterior segment outflow tissues in mice, the data presented in Examples 10-18, and their accompanying FIGS. 10-18D supports a determination as to whether direct enzymatic action by tPA is required for its effect on outflow facility or whether these effects are solely the result of receptor-mediated events. In the data below enzymatically inactive tPA was utilized.

In the examples that disclose enzymatically inactive tPA, the enzymatically inactive IPA (or variant thereof) can include one or more advantages for the treatment of IOP conditions, such as glaucoma. These advantages are noted below:
Less potential toxicity: tPA is a serine protease. As such it has enzymatic activity and can degrade many cell and extracellular matrix proteins. For this reason, it is tightly regulated both at the transcriptional level as well as through specific protein inhibitors (serpins) as well as binding proteins. Overproduction of tPA or exogenous supplementation can overwhelm the inhibitory action of these proteins allowing tPA to degrade potentially useful proteins that can adversely affect the eye tissue physiology. Upregulation of enzymatically inactive IPA allows tPA to continue to function as a cytokine while preventing it from proteolytically degrading other proteins.
Smaller sized molecule: Enzymatically inactive tPA engineered by deletion of part or all of its catalytic domain can decrease the size of the protein allowing a smaller sized transgene encoding for it to be packaged in a number of vectors that cannot accommodate packaging of the full length tPA.
Difference in bioavailability: Because serpin binding can be affected by deletion of part of the catalytic domain of tPA, enzymatically inactive tPA can be specifically engineered to have different (slower) elimination and thus higher bioavailability. Such a protein can also penetrate biological barriers more easily because of its smaller size.

The enzymatic activity of tPA has been well characterized and is dependent on the presence of an active site serine in position 478. Conversion of the active site serine-478 to an alanine reduces and/or removes the protein's enzymatic activity but still allows tPA to bind to receptors (i.e., LRP-1), inhibitors (i.e., PAI-1) and ligands (i.e., plasminogen).

To complement existing molecular tools, a clone of the enzymatically inactive version of sheep PLAT was created by site directed mutagenesis. To confirm that it encoded for a protein lacking enzymatic activity, cultured confluent human microvascular endothelial cells (HMVECs) were transfected and assayed, in the supernatant, the ability of tPA to convert plasminogen into plasmin. Transfection with the non-mutant sheep PLAT significantly increased tPA activity in the HMVEC supernatant in comparison to transfection with the enzymatically inactive mutant sheep PLAT. Given the relatively low transfection efficiency with such large plasmids, such differences were significant.

Since transfection of outflow pathways with plasmids is challenging, adenoviral transfection is utilized to achieve transgene expression in these tissues. Adenoviral transfection is effective in achieving at least short term (1-2 weeks) expression of transgenes in the TM and has been utilized for that reason. Although adenoviral injections in the anterior chamber can elicit a significant inflammatory response, concurrent use of steroids alleviates this effect and allows robust transfection and transgene expression. The use of an adenoviral vector carrying a fluorescent protein (mCherry), as well as the molecular quantification of the transgenic transcript by qRT-PCR, allowed for confirmation of successful transfection of TM cells in vivo.

Using this approach, it was confirmed that PLAT overexpression in the TM abrogates steroid-induced outflow facility reduction in mice. The mutant PLAT that encoded for a protein without enzymatic activity was equally effective in restoring outflow facility in steroid-treated mouse eyes.

To further confirm that the effect seen using adenoviral transfection is mediated via the tPA protein, another administration (in another set of animals) either enzymatically active or enzymatically inactive tPA was provided. As with the virally transfected eyes, both enzymatically active and enzymatically inactive tPA completely restored outflow facility that was reduced by steroid treatment.

Both enzymatically active and enzymatically inactive tPA (either by transfection or by direct protein supplementation) caused similar upregulation of Mmp-2, Mmp-9 and Mmp-13 expression in steroid treated eyes. Interestingly, these Mmps were upregulated to levels even higher than those detected in naïve (not exposed to steroid) mouse eyes. These findings indicate that the classic fibrinolytic serine protease activity of tPA is not critical for its effects on aqueous humor outflow regulation. Given that plasminogen expression has been shown to be below detectable limits in angle ring tissues containing the TM, it is unlikely that tPA regulates outflow facility via enzymatic activity, thus suggesting that tPA functions as a cytokine to induce changes in Mmp transcription, which ultimately results in the modulation of outflow.

To further confirm that tPA acts via a non-enzymatic mechanism to affect outflow facility, PlatKO mice were utilized. These mice have undetectable tPA enzymatic activity and display a significant reduction in outflow facility compared with wildtype littermates. Intravitreal administration of IPA in these animals increased outflow facility to levels similar to those of C57BL/6J mice (the background strain on which PlatKO mice are maintained). Enzymatically inactive tPA was equally effective in improving outflow facility in these animals. Administration of both enzymatically active and inactive tPA significantly increased Mmp-9 levels in the outflow tissues of these animals. In addition, enzymatically active tPA caused an upregulation (to a different degree) in expression of Mmp-13. Such changes have also been reported for tPA in other tissues.

The relationship between tPA and MMP-9 has been studied in the context of its therapeutic dosing for acute thrombotic cerebrovascular events. tPA administration results in enhanced MMP-9 activation that can lead to significant adverse outcomes. However, in the context of aqueous flow, upregulation or activation of Mmp-9 may have beneficial effects. To determine whether tPA induced Mmp-9 expression upregulation is critical for mediating its effects on outflow facility regulation, Mmp-9 deficient (Mmp-9KO) mice were utilized.

These mice were originally described in studies of carcinogenesis and are viable and fertile. Although they have diminished neuroretinal degeneration, they show no obvious (clinical) ocular phenotype. They do have significantly higher IOP than their wildtype littermates, This IOP elevation has been confirmed in comparison to C57BL/6J mice. Furthermore, this IOP elevation is caused by a significant reduction in outflow facility, corroborating previous findings on aqueous turnover in these mice. Intravitreal administration of either enzymatically active or enzymatically inactive tPA failed to increase outflow facility in these animals suggesting that Mmp-9 functions downstream of tPA to affect aqueous outflow and confirming the role of Mmp-9 in the regulation of outflow facility.

Given the detrimental effects of tPA induced MMP-9 upregulation in the brain, whether tPA increases Mmp-9 expression under baseline conditions (in the absence of steroids) potentially resulting in an excessive increase in outflow facility that could lead to hypotony was explored. Surprisingly, administration of tPA (either enzymatically active or enzymatically inactive) in C57BL/6J mice did not affect outflow facility nor Mmp-9 expression suggesting that its actions may be regulated by availability of its receptor or other downstream regulatory molecules.

The below findings in examples 10-18 provide a mechanism by which tPA is able to regulate outflow of aqueous humor following exposure to steroids. IPA likely functions as a cytokine to bind to a cell surface receptor and alter downstream intracellular signaling, as evidenced by the equal efficacies of enzymatically active and enzymatically inactive tPA in reversing steroid-induced outflow facility reduction in C57BL/6J mice and PlatKO mice. These physiologic effects are correlated to the enhanced expression of Mmp-9, which likely occurs via downstream transcriptional regulation as evidenced by the inability to enhance outflow facility in Mmp-9KO mice.

Previous studies in other tissues have shown that MMP-13 may participate in the proteolytic activation of pro-MMP-9. If this is indeed the mechanism of action of Mmp-13 in the mouse outflow system, it would explain why outflow facility is reduced in Mmp9 KO animals even if Mmp-13 expression may become upregulated as a result of tPA or NE-tPA treatment. There is evidence indicating that the MMP-2 present in the aqueous humor remains inactive in its pro-peptide form and as such may not directly affect the outflow facility process.

LRP-1 is a potential receptor candidate linking extracellular tPA and Mmp transcription as it has been reported in other organ systems and is expressed in TM cells. LRP-1 is a scavenger receptor most classically linked to receptor-mediated endocytosis in lipoprotein metabolism. Beyond this role, however, LRP-1 is associated with downstream intracellular signaling cascades and has a high affinity for binding tPA. Both enzymatically active and enzymatically inactive tPA have been shown to bind to cell surface LRP-1, in complex with NMDA-R, to initiate its phosphorylation and subsequent extracellular-signal related kinases 1/2 (ERK1/2) activation leading to downstream gene expression changes including MMP-9.

In addition, LRP-1 has been reported to mediate endocytosis and degradation of excess tPA. Such an action may explain the fact that exogenously applied tPA (either enzymatically active or enzymatically inactive) failed to increase outflow facility or alter Mmp expression under baseline conditions in C57BL/6J mice. Also, another member of the LRP gene family (LRP-2), which is also a potential receptor for tPA, has been implicated in glaucoma pathophysiology as it has been reported that lrp2 deletion causes significant IOP elevation in zebrafish.

In Examples 10-18 it is shown that tPA enzymatic activity is not essential for its action on regulating aqueous outflow and that its action is at least in part mediated by transcriptional control of Mmp-9. Previous studies on the role of intraocular MMP-9 indicate that its expression is necessary for proper collagen turnover at the iridocorneal angle. Although tPA-dependent Mmp expression upregulation does not prove subsequent enzymatic activity at the TM, it suggests such a mechanism of action. Furthermore, tPA (either enzymatically active or inactive) has little or no effect on outflow facility in mice under baseline conditions, making it useful for treating elevated IOP in steroid-induced glaucoma.

In Example 19 below, the transgenic mouse model having the human MYOC gene modified to contain the Y437H mutation (Tg-MYOCY437H) was utilized to determine whether tPA can improve outflow facility independent of its effect on steroid-induced outflow facility reduction. This effect was compared to that of ER stress modulator sodium phenylbutarate (PBA). Steroids were further tested to determine whether they further reduce outflow facility in these animals. It was also determined whether tPA affects Mmp expression levels in Tg-MYOCY437H mice.

Primary Open Angle Glaucoma (POAG) has been associated with genetic polymorphisms in a large number of loci. However, despite an apparent strong genetic component, few individual genes have a direct causative role for the disease. One of these genes is MYOC which encodes for the myocilin protein. Mutations or polymorphisms in the myocilin gene account for ˜4% of the cases of POAG and appear to often cause an aggressive disease that is characterized by IOP elevation early in life.

The myocilin protein was initially described in trabecular meshwork (TM) cell culture following glucocorticoid stimulation but is ubiquitously expressed in most body tissues, as well as the eye. Steroids not only cause an upregulation in myocilin expression, but also its secretion in the TM. This increased expression however, does not appear to be causative for steroid-induced IOP elevation.

Various mutations in the myocilin molecule cause disease of variable severity. The majority of glaucoma-associated MYOC mutations occur within exon 3, which encodes the olfactomedin-like domain. Several of these mutations prevent proteolytic cleavage at this myocilin domain, within the endoplasmic reticulum (ER), which is necessary for its extracellular secretion into the aqueous humor. This causes the accumulation of insoluble misfolded proteins within the ER leading to alterations in normal TM cell function and reducing phagocytic debris removal, ultimately resulting in TM cell loss. One of the mutations associated with severe IOP elevation early in life is the Y437H mutation. This missense mutation has been shown to prevent proper protein folding within the ER, which hinders progression through the secretory pathway.

Outflow resistance is highly dependent on the extracellular matrix (ECM) deposited by TM cells. Along with the intracellular ramifications of mutant myocilin, there are also changes to the extracellular environment of the outflow pathways. Since wildtype myocilin normally forms a dynamic network with several components of the TM ECM, such as fibrillin, laminin, collagen and fibronectin, mutations in myocilin cause an imbalance in this network and promote TM structural abnormalities. MYOC mutations have also been linked to ultrastructural TM changes in human tissues, including thickened basement membranes and apoptotic TM cells. In addition, mutant myocilin is associated with a reduction in the activity of matrix metalloproteinases (MMP-2 and MMP-9) in cultured TM cells. The death of TM cells in the presence of over-accumulation of mutant myocilin in their ER, likely exacerbates the ECM turnover deficiency.

Steroid-induced IOP elevation is another condition associated with changes in the ECM of the TM. Some mechanisms involved in abnormal ECM deposition in steroid-induced glaucoma have been studied, and there is an important role for tPA in ECM turnover regulation. It has been shown that absence of tPA leads to a decrease in outflow facility in mice in the absence of steroids. This implies that tPA (and potentially other fibrinolytic enzymes) may have a larger role in outflow facility regulation in glaucoma. Example 19 is directed to this question, wherein a mouse model of OAG that closely mirrors the well characterized human “myocilin” glaucoma was utilized. The transgenic mouse model containing the human MYOC gene modified with the Y437H mutation (Tg-MYOCY437H) displays several glaucoma phenotypes, including: IOP elevation, retinal ganglion cell death and optic nerve axon degeneration. It was confirmed that IOP is elevated in these animals early in life and that this is related to a significant reduction in outflow facility.

In Example 19, it was initially explored whether fibrinolytic enzyme expression in Tg-MYOCY437H mice is reduced. Despite expectations to the contrary, expression of both tPA, uPA and their inhibitor (PAI-1) were not affected. This finding suggests that at least early in life, TM cells do not experience feedback inhibition on fibrinolytic enzyme transcription. However, since activation of both tPA and uPA requires processing within the ER it is possible that their activity is decreased. Enzymatic activity of fibrinolytic enzymes in Tg-MYOCY437H mouse eyes were not checked.

Despite a seemingly unaffected fibrinolytic system in the outflow tissues of Tg-MYOCY437H mice, significantly lower expression of Mmp-2 and Mmp-9 was detected. Lower expression of Mmps could explain the reduction in aqueous humor outflow in these mice by disrupting ECM turnover.

It was then determined whether Tg-MYOCY437Hmice develop steroid-induced outflow facility reduction. Steroid-induced IOP elevation occurs in a very high percentage of patients with OAG. Contrary to expectations, no further outflow facility reduction inTg-MYOCY437H mice exposed to steroids was detected, however typically mice do show a robust steroid-induced effect on outflow facility. A possible explanation may involve the already reduced baseline Mmp expression in the transgenic mice as prior studies have shown steroid-induced TM changes occur due to MMP expression downregulation.

tPA has been shown to prevent and reverse steroid-induced outflow facility changes in mice and to affect sheep in a similar way. It has been shown that tPA reverses Mmp changes induced by steroids. Thus, a reasonable hypothesis could be made that an upregulation in Mmp expression could enhance outflow facility in Tg-MYOCY437H mice. The results below demonstrate that intraocular administration of tPA can partially reverse outflow facility reduction in Tg-MYOCY437Hmice. To determine whether this effect is dependent on enzymatic activity or receptor-mediated transcriptional upregulation, the ability of enzymatically inactive tPA to improve outflow facility in Tg-MYOCY437H mice was also tested. Enzymatically inactive tPA (NE-tPA/S478A-tPA) maintains the cytokine functions of tPA while completely abolishing its enzymatic activity. It has been shown that both enzymatically active and enzymatically inactive forms of tPA are equally effective in reversing steroid-induced outflow facility reduction in C57BL/6J mice (submitted) and that this effect is mediated through transcriptional control of Mmp-9. This effect was replicated in Tg-MYOCY437H mice, where both tPA and NE-tPA significantly improved outflow facility by ˜31% and ˜37%, respectively. The fact that outflow facility did not improve to levels seen in wildtype animals is understandable given the short duration of these experiments as well as the fact that Mmp-9 dysregulation may not be the only change that affects the outflow of the Tg-MYOCY437H mice.

It is however telling that enzymatically inactive tPA (NE-tPA) caused upregulation of both Mmp-9 and Mmp-13 expression, while enzymatically active tPA treatment caused Mmp-13 expression upregulation following a 2 day treatment regimen. Although a significant increase in Mmp-9 expression 2 days after tPA administration was not detected, it was detectable 5 days post administration, suggesting that such expression increase may also be present earlier albeit at a lower level.

Though tPA was successful in enhancing outflow facility, it is of course unlikely that it significantly affects the underlying pathologic events in the Tg-MYOCY437H mouse eyes. Since myocilin mutations ultimately lead to glaucoma through a significant increase in IOP (and concomitant reduction in outflow facility) it is important to compare the efficacy in improving outflow facility by targeting a downstream event vs. targeting a key pathophysiologic event.

ER stress modulation has been previously shown to result in lower IOP and prevent optic nerve degeneration and retinal ganglion cell death in this mouse model of “myocilin” glaucoma. Phenylbutyric acid (PB A) has been used as it acts as a chemical chaperone to reduce ER stress. Both systemic and topical administration were effective in reducing IOP in Tg-MYOCY437H mice. Topical application twice daily resulted in measurable IOP changes within a period as short as 1 week. Whole eye outflow facility was used as the metric because it has been shown to be more sensitive for detection of changes at the TM and provides a more reliable view of AH dynamics in human eyes.

To compare the efficacy of tPA administered by intraocular injection with that of topical PBA, the measurement of outflow facility 5 days after initiation of treatment was selected. tPA effectiveness should progressively decrease as medication concentration declines following bolus intraocular administration. Conversely, the effect from topical PBA would slowly increase as cells function better under a reduced ER stress. Thus this time-point for measuring OF appeared to be a reasonable compromise in obtaining an effect from both tPA and PBA.

As expected, both PBA treatment and tPA treatment resulted in an improvement of outflow facility. It appears that both treatments were comparable in their effectiveness at attenuating outflow facility reduction in Tg-MYOCY437H mice. While PBA primarily functions by reducing ER stress, it also caused an upregulation in Mmp-9 expression in treated eyes. tPA caused a similar increase in Mmp-9 expression. Furthermore tPA treatment resulted in a significant increase in Mmp-13 expression that surpassed expression levels in both PBA treated eyes and naïve wildtype eyes.

In summary, in Example 19, tPA expression is not reduced in Tg-MYOCY437H mice, and these mice do not show any appreciable steroid-induced outflow facility reduction. However, tPA is effective in improving outflow facility in this genetic POAG model. Furthermore, the tPA effect is accomplished in a receptor-mediated fashion and does not rely on tPA enzymatic activity. Finally, tPA appears to be equally effective with PBA in improving outflow facility in these mice such that it can have a therapeutic potential in the treatment of OAG. This action seems to involve upregulation of Mmps.

The present disclosure is further illustrated by the following non-limiting examples.

### EXAMPLES

### Example 1. Treatment of Mice with Gene Therapy

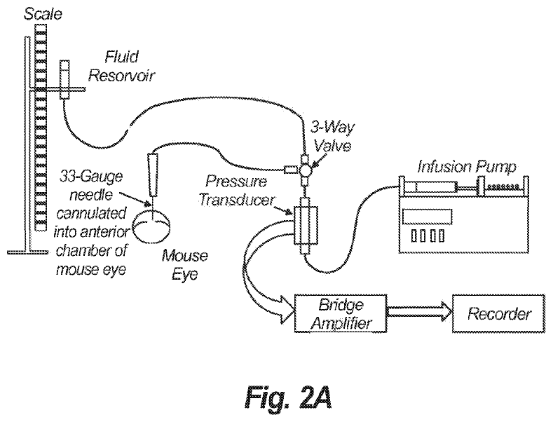

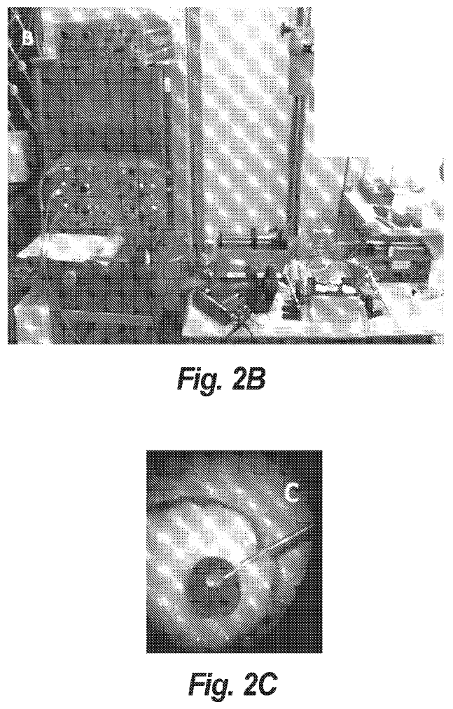

Methods: Adenoviral vectors carrying cDNA of the sheep PLAT gene and a fluorescent reporter gene (mCherry) (AdPLAT) or with no transgene (AdNull) were created. Transgene expression was driven by the CMV promoter. 3 groups of C57/B6 mice received either: (1) 20 μl of triamcinolone acetonide (TA) suspension (40 mg/ml) subconjunctivally bilaterally followed immediately by unilateral intracameral injection with 2 μl AdPLAT (3-4×10¹² VG/ml); (2) 20 μl TA subconjunctivally bilaterally followed one week later by unilateral intracameral injection with 2 μl AdPLAT; or (3) 20 μl TA subconjunctivally bilaterally followed immediately by bilateral injection with 2 μl adenovirus AdNull. IOP was measured preterminally. Outflow facility was determined using simultaneous pressure and flow measurements (see FIGS. 2A-2C). After outflow facility measurement, all AdPLAT injected eyes were dissected and viewed under a fluorescent microscope to inspect for mCherry expression in the trabecular meshwork (TM). Eyes that showed mCherry expression were analyzed as a separate group from eyes that showed minimal mCherry expression.

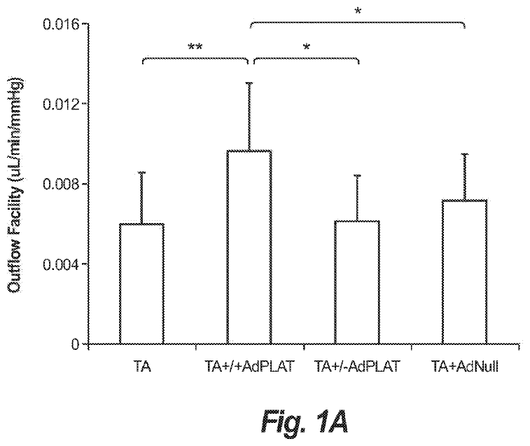

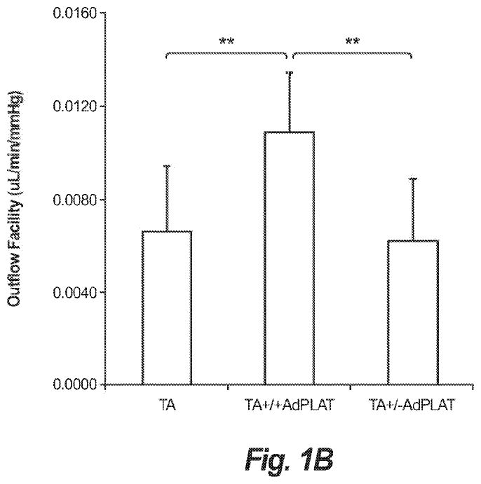

Results. IOP was not significantly different between all eye groups (p>0.05) after either one or two weeks of treatment with TA steroid. Eyes subjected to one week of TA treatment that showed mCherry/PLAT expression (TA+AdPLAT) had 63%, 54%, and 31% higher outflow facility than AdPLAT treated eyes with minimal mCherry/PLAT expression (TA+/−AdPLAT), contralateral control eyes, and AdNull treated eyes, respectively. In animals injected with AdPLAT concurrently with TA, the mean±standard deviation (SD) outflow facility in AdPLAT injected eyes showing robust mCherry expression (TA+/+AdPLAT) (n=12), AdPLAT injected eyes showing minimal or no mCherry expression (TA+/−AdPLAT) (n=8), and contralateral eyes injected with TA alone (TA) (n=22) was 0.0097±0.0033, 0.0062±0.0022, and 0.0060±0.0025, respectively, while eyes that received both TA and AdNull concurrently (TA+AdNull) (n=30) had outflow facility of 0.0071±0.0023 (ANOVA, p<0.05). See FIGS. 1A-1B.

Eyes subjected to two weeks of TA treatment that showed mCherry/PLAT expression had 86% and 58% higher outflow facility than AdPLAT treated eyes with minimal mCherry/PLAT expression and contralateral control eyes respectively. In animals receiving AdPLAT one week after TA injection, the mean±SD outflow facility in TA+/+AdPLAT (n=6), TA+/−AdPLAT (n=11) and TA (n=24) eyes was 0.0109±0.0026, 0.0062±0.0027, and 0.0067±0.0028, respectively (ANOVA, p<0.05). See FIGS. 1A-1B.

Discussion Mice develop a steroid induced outflow facility reduction when treated with triamcinolone. The inventors used this animal model to test the effectiveness of tPA to prevent and reverse these changes. To deliver tPA in the trabecular meshwork the inventors generated an adenoviral vector (AdV) that carries the sheep tPA gene (PLAT) together with Histone2b tagged with a fluorescent protein (mCherry) under the control of CMV promoter. The inventors injected the AdV in mouse eyes either concurrently or 1 week after periocular injection of triamcinolone acetonide. Animals were sacrificed 1 week after AdV injection and outflow facility was determined. The eyes were then examined for expression of mCherry-H2B. Eyes with visible expression of mCherry (which is a surrogate for PLAT expression) showed a statistically significant (p<0.05) increase in outflow facility compared with eyes receiving a null AdV and eyes receiving the active AdV but without mCherry expression (FIG. 1A). This was true for both mice receiving the AdV concurrently as well as 1 week after triamcinolone administration (FIG. 1B). Some eyes (both with and without mCherry expression) showed mild corneal clouding.

Thus, treatment with AdPLAT can both prevent an increase in outflow facility caused by steroid treatment, as seen in FIG. 1A, and reverse a decrease in outflow facility caused by steroid treatment, as seen in FIG. 1B.

### Example 2. Treatment of Mice with Small Molecules

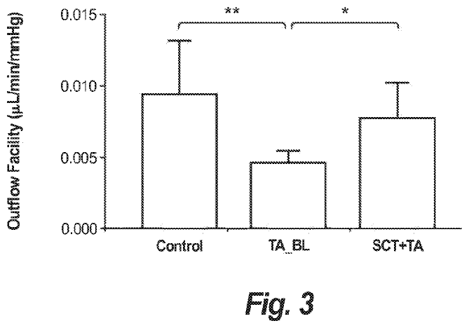

Mice were treated by gavage with a mixture of simvastatin, curcumin and troglitazone (“SCT”). Medications were selected for their direct and indirect effects on tPA. Treatment was initiated 5 days before the administration of steroids as in 1 above and continued for the duration of the experiment. One week after steroid administration outflow facility was measured as above. As seen in FIG. 3, outflow facility of eyes treated with the small molecule combination (SCT+TA) was similar to that of control eyes (Control) and significantly higher than that of steroid treated only eyes (TA_BL). Average+SD for SCT+TA group were 0.00776667±0.002408840 min/mmHg.

Thus, small molecule treatment can lower IOP and improve outflow facility.

### Example 3. Treatment of Sheep with Recombinant Human tPA

Methods. The inventors used recombinant human tissue plasminogen activator (rtPA) (ACTILYSE; Boehringer Ingelheim SA, Buenos Aires, Argentina) in two different protocols.

Protocol 1. 8 sheep of the Corriedale breed were treated with prednisolone acetate three times a day in both eyes, leading to elevated IOP. After one week, the animals received intravitreal injections of human recombinant tPA (100, 200, 500 and 1000 μg, two animals each) dissolved in balanced salt solution (BSS) in one eye. IOP was monitored for 19 more days while the animals continued to receive treatment with prednisolone. Periodic slit lamp examination was also performed.

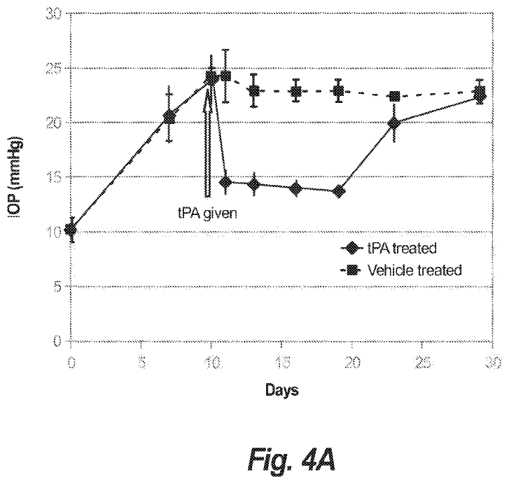

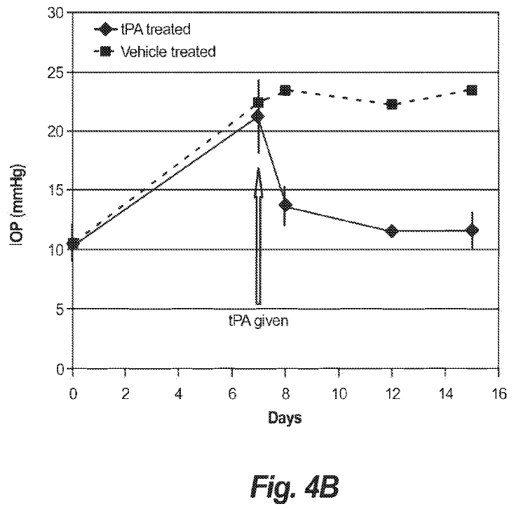

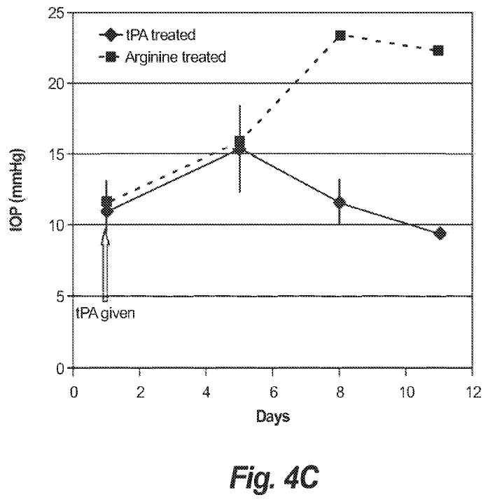

Results—Protocol 1. Sheep develop a well characterized IOP elevation after treatment with topical prednisolone acetate (Gerometta, R., et al., Invest Ophthalmol Vis Sci 50(2): 669-73 (2009)). This treatment is caused by a reduction in outflow facility. As seen in FIG. 4A, treatment with prednisolone for 10 days increased mean (±SD) IOP to 24.1 (±1.6) mmHg from a baseline of 10.2 (±1.1) mmHg (p<0.00001, t-test). Treatment with tPA decreased IOP within 24 h for all doses tested to 14.1 (±1.1) mmHg which was significantly lower than of the contralateral uninjected eye for all animals (p<0.00003, paired t-test). The effect was evident for all tPA doses, independent of the dose (p>0.05. ANOVA) and lasted for 19 days at which time IOP in the two eyes became similar (p>0.05). Transient injection and corneal clouding was observed in some eyes but was unrelated to the dose injected.

Protocol 2. In a second set of experiments, arginine was added to the BSS vehicle administered to the left eye to control for the relatively high concentration of the amino acid in the commercially available tPA lyophilisate. In these experiments all right eyes received 0.1 mg tPA, which concomitantly delivered 4.23 mg of arginine into the vitreous, and the left eye received this same amount of arginine, absent the tPA. On Day 1, prednisolone treatments were also begun simultaneously on both eyes.

Results—Protocol 2. Treatment with arginine alone failed to reduce outflow facility in the eye receiving arginine plus prednisolone (OS), compared to the eye treated with tPA and prednisolone (OD). See FIG. 4B.

### Example 4. Treatment of Sheep with tPA Prevents IOP Elevation

Methods. In this experiment, the inventor sought to detect the effects of tPA treatment on prevention of steroid-induced IOP elevation. In 4 sheep tPA was injected in one eye (100 μg) and they were started on prednisolone acetate three times a day in both eyes. Animals were monitored for 11 days on continuous treatment.

Results. As can be seen in FIG. 4C, rtPA prevented IOP elevation in the eyes treated with steroids when administered concurrent with steroid therapy. Transient injection and corneal clouding was observed in some eyes.

### Example 5. tPA Mediates Outflow Facility in the Absence of Steroid Treatment

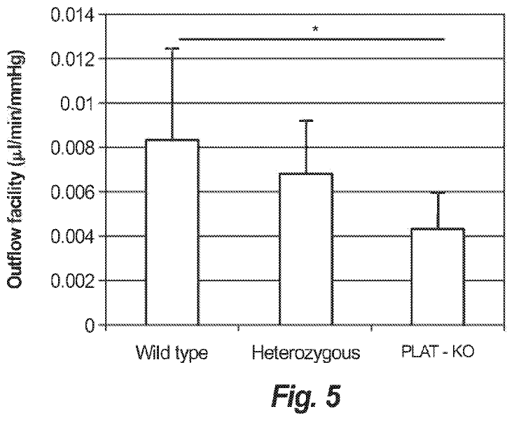

Results. To determine whether the above findings are relevant in the regulation of outflow facility, without the exogenous administration of steroids, and thus determine the physiological significance of tPA, the inventors determined outflow facility in tPA (PLAT)-knock out (KO) mice maintained in the C57BL/6 background (obtained from Jackson Laboratory, Bar Harbour, Maine). These animals develop normally, are fertile, and have a normal life span. They show no histological abnormalities, but pulmonary clot lysis is 21% that of normal wildtype siblings. Fibrin dissolution by PLAT-deficient macrophages is unaffected. The animals also have normal eyes with no evidence of media opacification. The inventors determined outflow facility in a small number of eyes from KOs, heterozygotes and wild-type littermates. Outflow facility of tPA (PLAT)-KO animals was only ˜50% of that of their wild type littermates (a statistically significant difference—ANOVA p<0.05, Tukey post hoc analysis), with heterozygotes having intermediate outflow facility values (FIG. 5).

Thus, IPA reduction reduces outflow facility, further supporting the use of tPA therapeutic agents to treat glaucoma and other IOP-related conditions.

### Example 6. MMP Expression is Affected by tPA Treatment

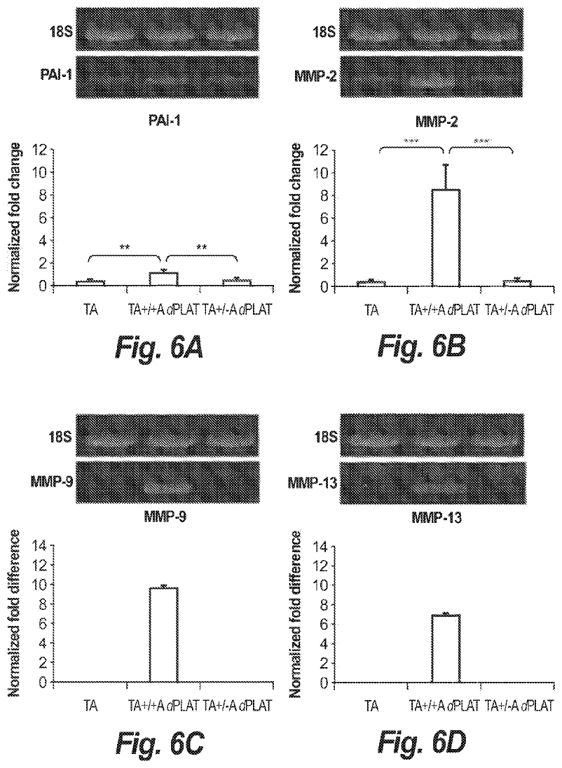

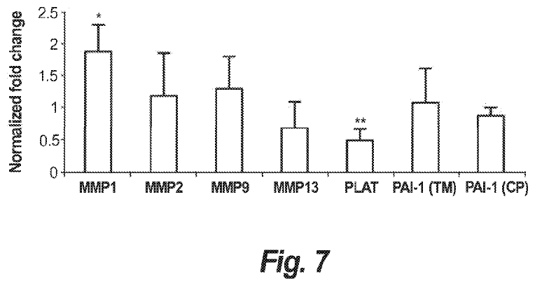

Results. The inventors determined the effects of tPA on the expression of matrix metalloproteinases (MMPs) in both the steroid-induced mouse outflow facility (FIGS. 6A-6D) and the sheep IOP elevation (FIG. 7) models. In mouse eye angle rings treated with steroid, expression of PAI-1, MMP-2, MMP-9, and MMP-13 is low; however, expression of these genes is increased in eyes that express AdPLAT (FIGS. 6A-6D). In sheep, administration of tPA up-regulates expression of MMP1, MMP2, MMP9, and MMP13, as well as up-regulating PAI-1 expression in the trabecular meshwork (TM) and the ciliary processes (FIG. 7). These data suggest that tPA action is at least in part mediated through MMP upregulation.

### Example 7. Gene Expression in Sheep Treated with tPA

Methods. Tissue Collection and Isolation of RNA. After animals were euthanized, eyes were immediately enucleated. Eyes were then opened anterior to the equator using a razor blade. The lens was removed from the anterior part, and the tissue was immersed in RNA stabilizing agent (RNAlater; Ambion, Carlsbad, CA) and placed at 20 8C for transportation to the United States. Upon arrival in the United States, TM and ciliary processes (CP) were dissected on ice in the presence of RNA stabilizing agent as described previously. Dissected tissue was homogenized, and total RNA was extracted using TRIzol reagent (Gibco. Carlsbad, CA). Briefly, the tissue was homogenized in TRIzol, and chloroform was added to separate proteins from RNA. After centrifugation, the RNA-containing supernatant was aspirated. The RNA was precipitated with isopropanol, washed with 75% ethanol, treated with DNase, and column purified using a commercial kit (RNAeasy Mini Kit; Qiagen, Valencia, CA) in accordance with the manufacturer's instructions. RNA concentrations were determined with a spectrophotometer (Nanodrop; Thermo Scientific, Wilmington, DE) and the 260:280-nm absorbance ratio was calculated to determine RNA purity.

Quantitative Real-Time PCR (qRT-PCR) The RNA samples were reverse transcribed with random hexamers to cDNA using a reverse transcription kit (Quantitect; Qiagen) in accordance with the manufacturer's instructions. Quantitative RT-PCR was performed using a commercial kit (SYBR Green RT-PCR Reagents Kit; Applied Biosystems, Carlsbad, CA) in an ABI PRISM 7900HT sequence detector (Applied Biosystems). The sheep endogenous mRNA expression of matrix metalloproteinase-1 (MMP-1), matrix metalloproteinase-2 (MMP-2), matrix metalloproteinase-9 (MMP-9), matrix metalloproteinase-13 (MMP-13). and plasminogen activator tissue (PLAT) in the TM were investigated. Plasminogen activator inhibitor 1 (PA1-1) mRNA expression was measured in both TM and ciliary processes tissues. The primer sequences used are listed in the Table. Relative quantification of gene expression was performed using the standard curve method. Mean threshold cycle (Ct) of the samples was compared among the groups by using the Ct. of 18S as an internal control. The DCt was calculated as the difference in Ct values derived from the target gene and the 18S gene. The DDCt was calculated as DCt of the normalized assayed genes in the treated samples minus DCt of normalized assayed genes in the naive control samples. Relative expression was calculated by the 2exp DD Ct formula.

Results. Administration of tPA up-regulates expression of specific matrix metalloproteinases (MMPs) MMP1 and MMP9, and down-regulates PLAT expression (FIG. 7).

### Example 8. PLAT is Upregulated Early after Steroid Application

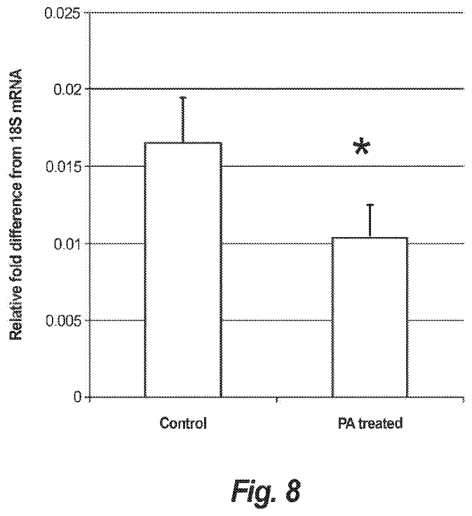

Results. The probability that PLAT expression is directly modulated by steroid is supported by the fact that the PLAT gene has multiple transcription factor binding sites. Bioinformatic analysis (DECODE—SABiosciences) reveals that both the human and mouse PLAT genes have binding sites for the glucocorticoid receptors (GR) (alpha and beta), API, CREB and NF-KappaB (all reported to act with GR to cause gene repression) within the genomic interval of 20 kB upstream and 10 kb downstream of the gene start codon, suggesting that such direct regulation is possible. In experiments in HTM cells from two donors, the inventors detected PLAT expression downregulation as early as 1 hour after prednisolone administration (FIG. 8). Thus, steroid treatment downregulates tPA. In addition, tPA downregulation represents a biomarker for the development of steroid-induced IOP elevation.

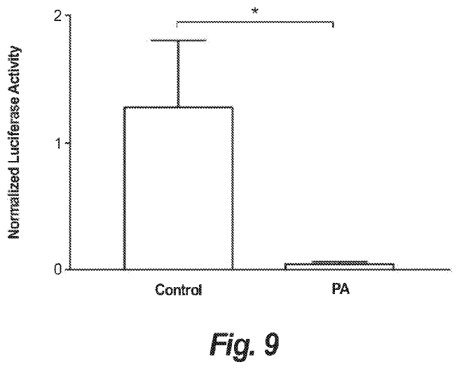

In addition, an additional study on HTM cells indicates that steroid regulation of PLAT is dependent on the first 800 bases proximal to the ATG site of PLAT (FIG. 9).

### Example 9. Administration of tPA Agents to the Anterior Chamber Reduces IOP

Methods. Lyophilized IPA, obtained as Acetilyse® from Boehringer Ingelheim S.A. (Buenos Aires) containing arginine. was used. Five sheep of the Coriedale breed were selected. Initially all eyes received instillation of 1% prednisolone 3 times/day for 10 days to elevate their IOP from 10 mm Hg to about 23 mm Hg. Then, 0.0001 μg was injected into one of the eyes and its effect was followed for up to 55:00 hrs while the instillation of prednisolone continued in both eyes. The same protocol was implemented for the 0.001 and 0.01 μg amounts (after extended washout) in the contra lateral eyes. Arginine, which is associated with 0.01 ug tPA, was injected alone and had no effect.

Results: Injection of 0.0001 lag into the AC had no effect on IOP of 23.0 mm Hg at 6:00 and 30:00 hrs after injection. 0.001 μg reduced IOP from 23.1 to 18.6 mmHg at 6:00 hr but IOP recovered to 22.3 mm Hg at 30:00 hr. Injection of 0.01 μg produced a marked and prolonged reduction of IOP. From a baseline of 23.4, IOP was reduced to 14.2, 19.0, 20.9, and 22.3 mm Hg at 6:00, 30:00, 48:00 and 55:00 hrs, respectively.

Thus, tPA is effective in reversing steroid-induced 10P elevation in sheep. The reduction of IOP elevation may be the result of an effect on extra-cellular matrix turnover in the TM. These findings further support the usefulness of tPA agents in the treatment of steroid-induced glaucoma.

In examples 10-18 below, the following methods were used:

Animals: 8- to 12-week-old female mice were used for this study. The animals were housed and bred at the State University of New York (SUNY) Downstate Health Sciences University Division of Comparative Medicine (Brooklyn. NY) under a 12-hour light/12-hour dark cycle and food ad libitum. C57BL/6J mice were obtained from The Jackson Laboratories (Bar Harbor, ME, USA). A PlatKO mouse colony was established from animals (stock No. 002508) obtained from The Jackson Laboratories. These animals are on a C57BL/61 background (www.jax.org/strain/002508). AnMmp-9KO mouse colony was established from animals (stock No. 007084) obtained from The Jackson Laboratories. These animals are on a mixed background but have been bred into the C57BL/6J background for five generations (www.jax.org/strain/007084). Protocols were approved by the SUNY Downstate Institutional Animal Care and Use Committee, and experiments were performed according to the ARVO Statement for the Use of Animals in Ophthalmic and Vision Research.

Adenoviral vector construction. The pShuttle-CMV-PLAT construct used has been previously reported and contains the full coding region of the sheep PLAT mRNA (1.8 kb) downstream of a CMV promoter and adjacent to a human histone 2B (H2B) tagged fluorescent reporter (mCherry) gene with internal ribosome entry site (IRES) at the multiple cloning site of the shuttle vector. To generate the non-enzymatic pShuttle-CMV-PLATNE construct, the PLAT gene insert was removed from the pShuttle-CMV-PLAT plasmid by restriction enzyme digestion and ligated into a pUC18 plasmid vector. Conversion of thymidine 1677 to guanosine (1677T>G) in the tPA enzyme active site was performed using the QuikChange Lightning Multi Site Directed Mutagenesis Kit, according to manufacturer's instructions (Agilent, Santa Clara, CA, USA). This caused alanine (GCG) to replace serine (TCG) at the catalytic site (S478A), rendering the resulting protein enzymatically inactive. The PLAT-NE construct was excised from the pUC18 vector and re-ligated into the pShuttle-CMV plasmid. All plasmids underwent restriction enzyme digestion to confirm proper fragment sizes and orientation. The inserts were directly sequenced to confirm the nucleotide sequence (GENEWIZ, South Plainfield, NJ, USA).

The loss of enzymatic activity in pShuttle-CMV-PLATNE was tested in comparison to pShuttle-CMV-PLAT via plasmid transfection, into 80% confluent human microvascular endothelial cells (HMVEC; ThermoFisher Scientific, Waltham, MA, USA) in a 12-well plate, through the use of Targefect-RAW (Targeting Systems, El Cajon, CA, USA). Transfection efficiency was visualized by intracellular fluorescent expression of mCherry. Supernatants were collected from all culture plate wells and enzymatic activity was assessed in a time dependent manner from 3-8 hrs via a Tissue type Plasminogen Activator Human Chromogenic Activity Assay Kit, per manufacturer's instructions (ab108905, Abcam Co, England). Results of chromogenic activity assay were normalized for total protein amount in samples, as determined by microBCA protein assay kit, per manufacturer's instructions (Pierce, ThermoFisher Scientific, Waltham, MA, USA).

Adenovirus vector carrying the non-enzymatic transgene (AdPLATNE) was generated by homologous recombination, amplification and purification by ViraQuest Inc (North Liberty. IA, USA). Frozen stocks of AdPLAT and AdNull (containing no transgene), that have been previously reported, were also used.

Steroid, adenoviral and protein injections. Intracameral adenovirus and intravitreal protein injections were performed using a 10 μl Hamilton syringe with 36-gauge stainless steel needles (WPI Inc. Sarasota, FL, USA) while subconjunctival injections were performed using a 100 μl Hamilton Syringe with 26-gauge needles (Precision Glide, Becton Dickinson & CO, Franklin Lakes, NJ, USA). All injections were performed under isoflurane inhalation anesthesia and topical anesthesia with 0.5% proparacaine. C57BL/6J mice undergoing adenovirus treatment received bilateral injections with 20 μl of triamcinolone acetonide (TA) suspension (40 mg/ml, Kenalog-40; Bristol-Myers Squibb, NY, USA) subconjunctivally immediately prior to the intracameral adenovirus injection. Animals were then divided into three groups (FIG. 10a):
1) Animals that received unilateral intracameral injection with 2 μl of AdPLAT suspension (1.1×10¹² vg/ml) while the contralateral eye received 2 μl of balanced salt solution (BSS; Alcon Laboratories Inc, Fort Worth, TX, USA),
2) Animals that received unilateral intracameral injection with 2 μl of AdPLATNE suspension (1.2×10¹² vg/ml) while the contralateral eye received 2 μl of BSS, and
3) Animals that received bilateral intracameral injection with 2 μl of AdNull suspension (1.1×10¹² vg/ml). Animals were euthanized one week after AdPLAT. AdPLATNE or AdNull treatment.

C57BL/6J, PlatKO and Mmp-9KO mice were treated with intravitreal tPA (either enzymatically active or enzymatically inactive) or bovine serum albumin (BSA). C57BL/6J mice were divided into two groups:
1) Animals receiving bilateral steroid (TA) injection subconjunctivally five days prior to protein injection (FIG. 10b) and
2) Animals not receiving subconjunctival steroids (FIG. 10c).

PlatKO and Mmp-9KO mice were also not given steroids prior to protein injections (FIG. 10c). Within each cohort, animals were divided into two groups:
1) Animals that received unilateral intravitreal injection with 2 μl of tPA (5 μg/μl, Actilyse; Boehringer Ingelheim, Ingelheim am Rhein, Germany) while the contralateral eye received 2 μl of BSA (5 μg/μl; Gold Biotechnology, St Louis, MO, USA), and
2) Animals that received unilateral intravitreal injections with 2 μl of non-enzymatically active tPA (NE-tPA/S478A-tPA) (5 μg/μl; Innovative Research, Novi, MI, USA) while the contralateral eye received 2 μl of BSA. Animals were euthanized two days after intravitreal injection.

IOP measurement: IOP was measured in Mmp-9KO mice pre-terminally with a rebound tonometer. Animals were held in a custom-made restrainer that does not compress the chest or neck, while IOP is measured. IOP measurements were performed after application of 0.5% proparacaine topical anesthesia. Five measurements were obtained per eye and averaged. IOP measurements were performed between 10 AM and 12 PM, to minimize the effect of diurnal IOP variation.

Outflow facility determination: Mouse eyes were enucleated immediately after euthanasia. Outflow facility was determined using a constant pressure method, as previously described. Pressure was raised in steps of 4 cmH₂O, from 8 cmH₂O (5.88 mmHg) to 32 cmH₂O (23.54 mmHg) by increasing the height of a column of fluid of BSS. Steady state was initially achieved after 10 minutes. Stabilization between all subsequent steps was obtained within 5 minutes. Flow was constantly measured via a microfluidic flow sensor (0.07-1.5 uL/min, MFS1; Elveflow, Paris, France). For analysis, flow rates at each pressure level were plotted and the slope of the regression line was used to calculate the outflow facility for each eye. Any eyes that developed visible leaks during outflow facility determination, or that had pressure-flow correlations with R²<0.9, were excluded from analysis but were used for RNA quantification.

Tissue collection and confirmation of transgene expression: After outflow facility determination in adenovirus treated mice, mCherry expression in the TM was determined in all AdPLAT and AdPLATNE injected eyes. The eyes were dissected on ice to isolate a rim of tissue containing the TM by removing most of the iris and ciliary body. Flat mounts of the rims containing TM were observed in an epifluorescent microscope equipped with the appropriate filter sets to visualize mCherry expression. After observation, dissected rims were immediately immersed in RNA stabilizing agent (RNAlater, Invitrogen by ThermoFisher Scientific, Waltham, MA, USA) and frozen. For TM tissue collection following protein treatment, all eyes were flash frozen in liquid nitrogen and subsequently dissected on ice to obtain the angle ring containing the TM tissues as previously described⁴³. Dissected TM tissues were immersed in RNAlater solution and then snap-frozen and stored at −80° C. until RNA extraction.

RNA isolation and quantitative real time PCR: Tissue collected was pooled (four eyes) and homogenized in TRIzol reagent (Life Technologies, Carlsbad, CA, USA). RNA was isolated per the manufacturer's instructions and resuspended in nuclease-free water. RNA concentration was determined with a Nanodrop ND-1000 Spectrophotometer (ThermoFisher Scientific, Waltham, MA, USA). cDNA was synthesized by using High-Capacity cDNA Reverse Transcription Kit (Applied Biosystems, Waltham, MA, USA) according to manufacturer's protocol. Quantitative real-time PCR (qRT-PCR) was performed by using Green-2-Go qPCR Mastermix-ROX (BioBasic, Amherst, NY, USA) on a QuantStudio 6 Flex thermal cycler (Applied Biosystems, Carlsbad, CA, USA).

mRNA expression of sheep PLAT and mouse Mmp-2, Mmp-9, and Mmp-13 in angle ring tissues was determined. Primers were designed by using Primer-BLAST (https://www.ncbi.nlm.nih.gov/tools/primer-blast/index.cgi?LINK_LOC=BlastHome; in the public domain) and their specificity was confirmed by the presence of a single band of the expected size on agarose gel electrophoresis. Primer sequences are listed in Table 1. Specificity was further verified during each experiment by inspection of melting curves to ensure the absence of multiple-sized amplification products. Annealing temperature was 60° C. Values of target mRNA expression were normalized to the expression levels of 18S (18S rRNA). The relative fold change was calculated by using the ΔΔCt method. The presence of outliers was tested by using the Thompson Tau test and any outliers were removed from analysis.

TABLE 1
Primer sequences of genes analyzed by qRT-PCR

| Number | Genc | Sequencc (5′-3′) |
|---|---|---|
| 1 | PLAT | FP: CAGTGCCCAGAAGGGTTCAT |
| | | RP: GTAGCACCAGGGCTTTGAGT |
| 2 | Mmp-2 | FP: ACAGTGACACCACGTGACAA |
| | | RP: GGTCAGTGGCTTGGGGTATC |
| 3 | Mmp-9 | FP: GCGTCGTGATCCCCACTTAC |
| | | RP: CAGGCCGAATAGGAGCGTC |
| 4 | Mmp-13 | FP: TACCATCCTGCGACTCTTGC |
| | | RP: TTCACCCACATCAGGCACTC |
| 5 | 18S | FP: AGTCCCTGCCCTTTGTACACA |
| | | RP: GATCCGAGGGCCTCACTAAAC |

FP, forward primer; RP, reverse primer

### Example 10: Confirmation of Lack of Enzymatic Activity of Cells Transfected with PLAT(1677T>G)

To confirm that the mutant PLAT (1677T>G) does not generate an enzymatically active tPA protein, cell culture supernatant from transfected confluent human microvascular endothelial cells (HMVECs; ThermoFisher Scientific, Waltham, MA, USA) was assayed for its ability to catalyze the conversion of plasminogen to plasmin. Transfection efficiency with these large plasmids (10.8 kb), based on the number of fluorescent cells, was approximately 30-40% and not significantly different between the pShuttle-CMV-PLAT and pShuttle-CMV-PLATNE transfected groups (p>0.05, t-test). The supernatant of cells transfected with pShuttle-CMV-PLAT had significantly different normalized enzymatic activity when compared to supernatant from pShuttle-CMV-PLATNE and that from non-transfected cells at all time points assayed (p<0.0001 for both group, time and their interaction, General Linear Model ANOVA)(FIG. 10D).

In FIG. 10D, tPA enzymatic activity in supernatants of non-transfected HMVECs and HMVECs transfected with either pShuttle-CMV-PLAT or pShuttle-CMV-PLATNE. IPA enzymatic activity in supernatant from pShuttle-CMV-PLAT transfected cells is significantly higher than the activity in supernatant from pShuttle-CMV-PLATNE transfected and that of non-transfected cells (p<0.0001, for both group, time and their interaction, General Linear Model ANOVA).

### Example 11: PLAT (and PLATNE) Expression in Adenovirus Injected Eyes

In animals injected with adenoviral vectors, mCherry expression was distributed uniformly along the entire length of the TM in AdPLAT (FIG. 11a) and AdPLATNE (FIG. 11b) treated eyes. Expression of the PLAT gene was detected by qRT-PCR in the TM of eyes receiving AdPLAT and AdPLATNE, respectively, while PLAT expression was below detection limits in naïve and AdNull treated eyes (FIG. 11c).

Flatmounts of anterior segments of mouse eyes from animal injected with (FIG. 11a) AdPLAT (TA+AdPLAT), and (FIG. 11b) AdPLATNE (TA+AdPLATNE). There is robust mCherry expression in both AdPLAT and AdPLATNE treated eyes. Arrows indicate mCherry-positive cells. (FIG. 11c) qRT-PCR quantification of PLAT expression in angle ring tissues. Levels of PLAT are detected in AdPLAT (n=16) and AdPLATNE (n=12) eyes, but are undetectable in AdNull (n=16) and naïve (n=20) eyes. TM=trabecular meshwork.

### Example 12: AdPLAT and AdPLATNE Attenuate Steroid-Induced Outflow Facility Reduction and Increase Mmp Expression

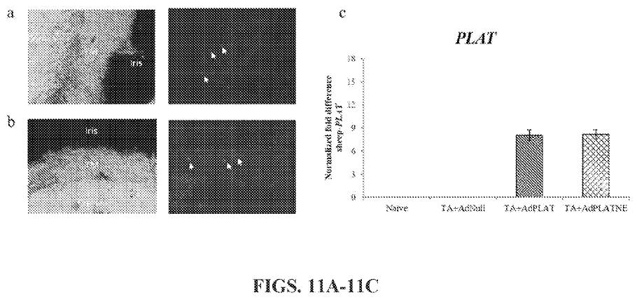

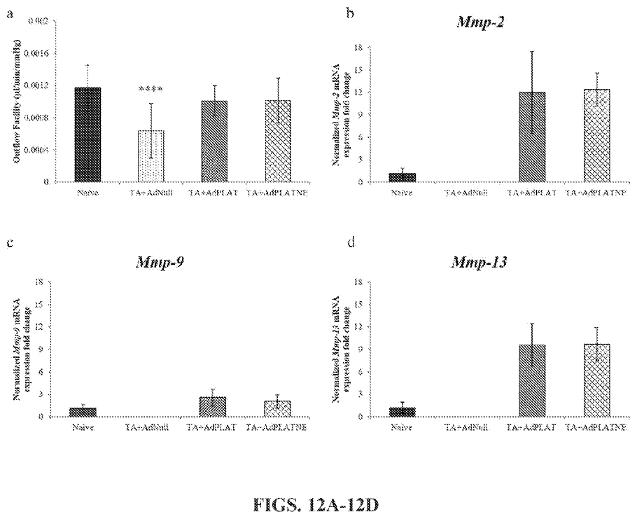

In eyes treated with TA and adenovirus, the mean±standard deviation outflow facility (μl/min/mmHg) was 100.9±18.7×10⁻⁵ in AdPLAT (n=11), 101.3±28×10⁻⁵ in AdPLATNE (n=8) and 63.6±33.8×10⁻⁵ in AdNull (n=11) treated eyes, while the outflow facility was 117±28.3×10⁻⁵ in naïve C57BL/6J eyes (n=10) (p<0.0001, ANOVA). Similar to previous reports, TA caused a ˜46% reduction in outflow facility from baseline while treatment with AdPLAT or AdPLATNE restored outflow facility to baseline levels in TA exposed eyes. AdNull treated eyes had significantly lower outflow facility than all other groups (p<0.0001, Tukey-Kramer post hoc analysis), while there was no significant difference between naïve, TA+AdPLAT or TA+AdPLATNE groups (p>0.05, Tukey-Kramer post hoc analysis) (FIG. 12a).

Expression of matrix metalloproteinases (Mmp-2, Mmp-9, and Mmp-13) in C57BL/6J eyes exposed to TA and AdNull (n=16), was below detection levels. Treatment with AdPLAT (n=16) and AdPLATNE (n=8) caused an upregulation in Mmp-2 (FIG. 12b), Mmp-9 (FIG. 12c) and Mmp-13 (FIG. 12d) expression (p<0.0001, p<0.001 and p<0.0001 respectively; ANOVA with Tukey-Kramer post hoc analysis). Mmp expression after AdPLAT and AdPLATNE appeared to be higher than that in naïve eyes (n=20).

In FIGS. 12a-12d, and FIG. 12a particularly, outflow facility in TA and Adenovirus treated C57BL/6J mouse eyes is shown. Outflow facility (mean±SD μl/min/mmHg) was significantly increased in eyes treated with TA+AdPLAT (n=11) and TA+AdPLATNE (n=8) compared with those treated with TA+AdNull (n=11) (****p<0.0001, ANOVA with Tukey-Kramer post hoc analysis). The outflow facility of naïve (not treated with either TA or adenovirus) C57BL/6 animals is included for comparison purposes. The outflow facility in these eyes is similar to the outflow facility in AdPLAT and AdPLATNE treated eyes. Gene expression changes in Mmp-2 (FIG. 12b), Mmp-9 (FIG. 12c), and Mmp-13 (FIG. 12d) were normalized (mean±SD) to values in naïve eyes. Expression was significantly different between TA+AdNull eyes (n=16) and both TA+AdPLAT eyes (n=16) and TA+AdPLATNE eyes (n=8). Naïve eyes (n=20) also had lower expression than the TA+AdPLAT and TA+AdPLATNE treated eyes. Asterisks indicate differences on Tukey-Kramer post hoc analysis, **p<0.01, ***p<0.001, ****p<0.0001. mRNA was below detectable limits for Mmp-2, Mmp-9 and Mmp-13 in the TA+AdNull group.

### Example 13: Both Enzymatically Active and Enzymatically Inactive Tissue Plasminogen Activator Attenuate Steroid-Induced Outflow Facility Reduction and Increase Mmp Expression

In eyes treated with TA and the respective protein injection, the mean±standard deviation outflow facility (μl/min/mmHg) was 67.8±25.8×10⁻⁵ in TA+BSA (n=18), 105.3±35.8×10⁻⁵ in TA+tPA (n=15) and 114±40.3×10⁻⁵ in TA+NE-tPA (n=10) (p<0.0001, ANOVA). Treatment with tPA or NE-tPA restored outflow facility to naïve baseline levels. There was a significantly lower outflow facility in TA+BSA eyes in comparison to that in all other groups (p<0.0001, Tukey-Kramer post hoc analysis). Furthermore, there was no significant difference between naïve, TA+tPA and TA+NE-tPA groups (p>0.05. Tukey-Kramer post hoc analysis) (FIG. 13a).

tPA (n=8) and NE-tPA (n=8) treated eyes showed a significant upregulation in Mmp-2 (FIG. 13b), Mmp-9 (FIG. 13c) and Mmp-13 (FIG. 13d) expression (p<0.0001, p<0.01 and p<0.01 respectively; ANOVA with Tukey-Kramer post hoc analysis) compared to BSA (n=4) treated controls and appeared to be higher than that in naïve eyes (n=20).

In FIGS. 13a-13d, and FIG. 13a particularly, outflow facility in TA and protein treated C57BL/6 mouse eyes is shown. Outflow facility (mean±SD μl/min/mmHg) was significantly increased in eyes treated with TA+tPA (n=15) and TA+NE-tPA (n=10) compared with those treated with TA+BSA (n=18) (****p<0.0001, ANOVA with Tukey-Kramer post hoc analysis). The outflow facility of naïve (not treated with either TA or protein) C57BL/6J animals is included for comparison purposes. The outflow facility in these eyes is similar to the outflow facility in tPA and NE-tPA treated eyes. Gene expression changes in Mmp-2 (FIG. 13b), Mmp-9 (FIG. 13c), and Mmp-13 (FIG. 13d) were normalized (mean±SD) to values in naïve eyes. Group means were significantly different in TA+BSA eyes (n=4), TA+tPA eyes (n=8), TA+NE-tPA eyes (n=8) (ANOVA, Mmp-2, p<0.0001, Mmp-9, p<0.0001, Mmp-13, p<0.01). Asterisks indicate differences on Tukey-Kramer post hoc analysis, *p<0.05, **p<0.01, ***p<0.001 ****p<0.0001.

### Example 14: Both Enzymatically Active and Enzymatically Inactive Tissue Plasminogen Activator Administration Rescues Outflow Facility in PlatKO Mice with an Upregulation in Mmp-9 and Mmp-13 Expression

In PlatKO mice receiving intravitreal BSA injections (n=19), the mean±standard deviation outflow facility (μl/min/mmHg) was 61±22×10⁻⁵. In agreement with previously reported results, these PlatKO mouse eyes had ˜48% reduced outflow facility compared to C57BL/6J eyes (n=10). Intravitreal administration of tPA or NE-tPA caused a 46% (89±27×10⁻⁵) and 51% (92±42×10⁻⁵) increase in outflow facility, respectively, compared to the outflow facility of BSA treated eyes. There was a significant difference in outflow facility among C57BL/6J naïve, PlatKO+BSA, PlatKO+tPA and PlatKO+NE-tPA eyes (p<0.001, ANOVA), with outflow facility in PlatKO+BS A eyes significantly lower in comparison to the two other groups (p<0.001, Tukey-Kramer post hoc analysis) (FIG. 14a).

Intravitreal tPA and NE-tPA administration in PlatKO mice caused a significant upregulation in Mmp-9 (FIG. 14c) expression (p<0.0001, ANOVA) compared to that in eyes treated with BSA (n=8) (p<0.001, p<0.0001, Tukey-Kramer post hoc analysis). There was also a significant upregulation in Mmp-13 (FIG. 14d) expression (p<0.0001, ANOVA) in tPA treated PlatKO eyes as compared to BSA treated eyes (p<0.001, Tukey-Kramer post hoc analysis). Mmp-2 (FIG. 14h) expression was not significantly different in tPA and NE-tPA treated PlatKO eyes compared to expression in BSA treated eyes (p>0.05, ANOVA).

In FIGS. 14a-14d, and FIG. 14a particularly, outflow facility in protein treated PlatKO mouse eyes is shown. Outflow facility (mean±SD μl/min/mmHg) was significantly increased in eyes treated with tPA (n=12) and NE-tPA (n=13) compared with those treated with BSA (n=19) (***p<0.001, ANOVA with Tukey-Kramer post hoc analysis). The outflow facility of naïve (not treated with protein) C57BL/6 animals is included for comparison purposes and is similar to the outflow facility in tPA and NE-tPA treated PlatKO eyes. Group means of gene expression changes in Mmp-2 (FIG. 14b), Mmp-9 (FIG. 14c), and Mmp-13 (FIG. 14d) were significantly different in PlatKO BSA eyes (n=8), tPA eyes (n=12), NE-tPA eyes (n=8) (ANOVA, Mmp-2, p<0.05, Mmp-9, p<0.0001, Mmp-13, p<0.0001). Asterisks indicate differences on Tukey-Kramer post hoc analysis, *p<_{0.05}, **p<0.01, ***p<0.001, ****p<0.0001.

### Example 15 Tissue Plasminogen Activator does not Alter Baseline Outflow Facility, Mmp or Plat Expression

Treatment with intravitreal tPA and NE-tPA did not cause further enhancement in outflow facility in C57BL/6J mouse eyes not previously exposed to steroids (p>0.05, ANOVA). The mean±standard deviation outflow facility (μl/min/mmHg) was 101.6±27.1×10⁵, 102.7±19.5×10⁻⁵ and 110±36.5×10⁻⁵ in BSA (n=19), tPA (n=11) and NE-tPA (n=11) treated eyes respectively (FIG. 15a). No significant changes in Mmp gene expression were detected between treatment groups (p>0.05, ANOVA) (FIGS. 15b-15d). Furthermore, exogenous tPA and NE-tPA did not cause significant changes in endogenous Plat expression (p>0.05, ANOVA) (FIG. 15e).

In FIGS. 15a-15e, and FIG. 15a particularly, outflow facility in protein treated C57BL/6 mouse eyes is shown. Outflow facility (mean±SD μl/min/mmHg) was unchanged following treatment in non-steroid treated eyes exposed to tPA (n=11) and NE-tPA (n=11) compared with those treated with BSA (n=19) (p>0.05, ANOVA). The outflow facility of naïve (not treated with protein) C57BL/6 animals is included for comparison purposes. The outflow facility in these eyes is similar to the outflow facility in tPA, NE-tPA or BSA treated C57BL/6 eyes. Group means of gene expression changes in Mmp-2 (FIG. 15b), Mmp-9 (FIG. 15c), and Mmp-13 (FIG. 15d) were not significantly different in BSA eyes (n=12), tPA eyes (n=8), NE-tPA eyes (n=8) (p>0.05, ANOVA).

### Example 16 Tissue Plasminogen Activator does not Rescue Outflow Facility Reduction in Mmp-9KO Mice

Similar to previous reports, intraocular pressure was significantly elevated (˜42%) in Mmp-9KO mice (n=32) when compared to C57BL/6J mice (n=28) (data not shown). Furthermore, mean±standard deviation outflow facility (μl/min/mmHg) was significantly reduced in Mmp-9KO mice (51.4±19.9×10⁻⁵; n=14) compared to C57BL/6J mice (124.4±29.2×10⁻⁵; n=16) (p<0.0001. T-test) (FIG. 15a). Treatment with intravitreal tPA or NE-tPA did not increase outflow facility in Mmp-9KO mouse eyes (p>0.05, ANOVA) (FIG. 15b).

In FIGS. 15a-15e, and FIG. 15a particularly, outflow facility in Mmp9KO and C57BL/6 mouse eyes is show. Outflow facility (mean±SD μl/min/mmHg) in Mmp9KO mouse eyes (n=14) was significantly lower than that of C57BL/6J animals (n=16) (****p<0.0001, T-test). In FIG. 15b outflow facility in BSA, tPA and NE-tPA treated Mmp9KO mouse eyes is shown. Outflow facility (mean±SD ill/min/mmHg) was not significantly different across groups Mmp-9KO+BSA (n=21), Mmp-9KO+tPA (n=13) and Mmp-9KO+NE-tPA (n=8). (p>0.05. ANOVA).

### Example 17 Enzymatically Active and Non-Enzymatically Active Tissue Plasminogen Activator Reverse Steroid-Induced Outflow Facility Reduction and Increase MMP Expression in HTM Cells

HTM cells were treated with 300 nM prednisolone acetate (PA) for 72 h to reduce outflow facility. They were then treated with either tissue plasminogen activator (tPA), non-enzymatically active tPA (NE-tPA) or bovine serum albumin (BSA) and perfused in artificial conventional outflow system (ACOS). Simulated outflow facilities (mean±standard deviation) (μl/min/mmHg/mm²) was 2.4±0.6×10-1 for PA, 3±1.2×10-1 for PA+BSA, 7±1.2×10-1 for PA+tPA and 5.4±0.9×10-1 for PA+NE-tPA (p<0.0001, ANOVA). Baseline outflow facility was 8.9±1.7×10-1 and was significantly different from all other treatment groups (p<0.0001, Tukey-Kramer post hoc analysis).

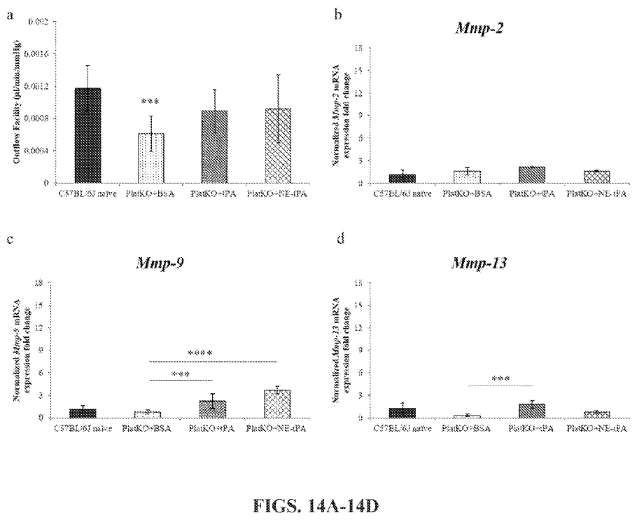

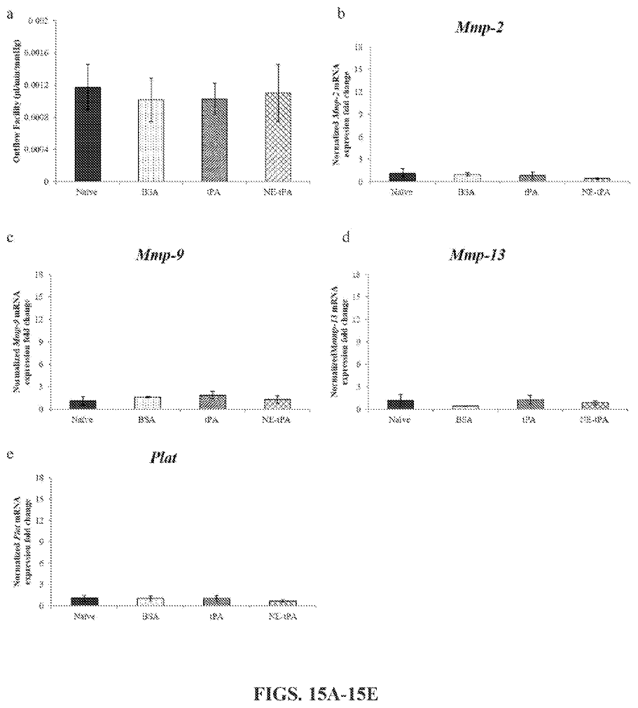

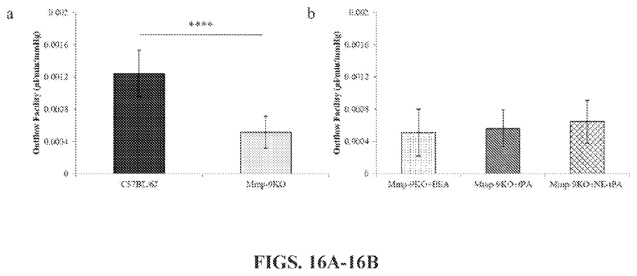

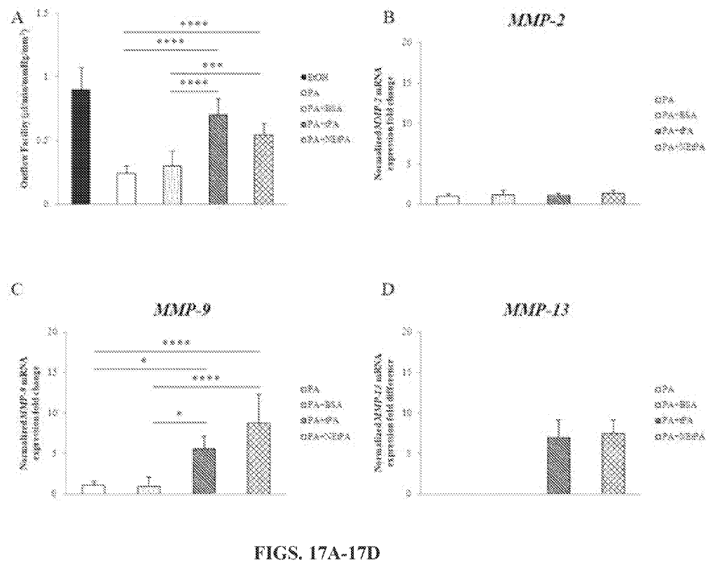

PA treatment caused a˜73% reduction in outflow facility compared to baseline, while PA+BSA treatment caused a˜66% reduction in outflow facility. Treatment with tPA significantly enhanced outflow facility compared to PA and PA+BSA levels (p<0.0001 and p<0.0001, respectively; Tukey-Kramer post hoc analysis). NE-tPA treatment resulted in similar improvements (p<0.001 and p<0.0001, respectively; Tukey-Kramer post hoc analysis) (FIG. 17A). tPA and EI-tPA treated HTM cells showed a significant upregulation in expression compared to PA+BSA treated cells (p<0.0001, ANOVA with Tukey analysis) (FIG. 17C). Expression of and NE-tPA treatment, respectively, but was below detection limits in PA and PA+BSA treated cells (FIG. 17D). MMP-2 expression was not significantly different between treatments (p>0.05, ANOVA) (FIG. 17B).

As further illustrated in FIGS. 17A-17D, outflow facility in PA treated HTM cells. Outflow facility (mean±SD in μ1/min/mmHg/mm 2) was significantly increased in cells treated with PA+tPA and PA+NE-tPA compared with those treated with PA alone or PA+BS A (****p<0.0001 ANOVA, Tukey-Kramer post hoc analysis). The outflow facility of EtOH treated (not treated with PA) HTM cells is included for comparison purposes in FIG. 17A.

Outflow facility of this group is significantly different from all other groups. Gene expression changes in MMP-2 (FIG. 17B), MMP-9 (FIG. 17C), and MMP-13 (FIG. 17D) are normalized (mean±SD) to values in PA treated cells. MMP-9 expression was significantly different between PA and PA+BSA treated cells and both PA+tPA and PA+NE-tPA treated cells. (ANOVA, MMP-2, p>0.05, MMP-9, p<0.0001). *p<0.05. **p<0.01, ***p<0.001, ****p<0.0001. mRNA was below detectable limits for MMP-13 in the PA and PA+BSA treated cells.

### Example 18: LRP-1 and NMDA-R Inhibition Reduce NE-tPA Mediated Outflow Facility Increase and Prevent MMP Expression in HTM Cells

HTM cells were treated with PA+NE-tPA and inhibitors of either LRP1 (RAP) or NMDA receptor (MK-801) and were perfused in ACOS. Simulated outflow facility (mean±standard deviation μl/min/mmHg/mm²) was 5.5±0.7×10-1 for PA+NE-IPA, 2.9±1.3×10-1 for PA+NE-tPA+MK-801, 2.7±1.2×10-1 for PA+NE-tPA+RAP and 3.3±1.3×10-1 for PA+NE-tPA+MK-801+RAP.

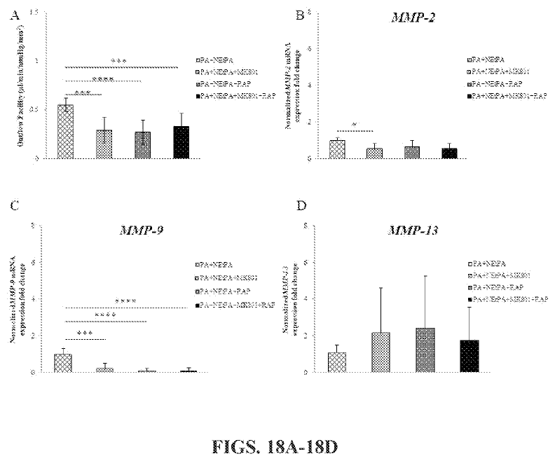

In treated groups, outflow facility was significantly lower across all HTM cells receiving either inhibitor or their combination compared to PA+NE-tPA treated cells (p<0.0001, ANOVA, Tukey-Kramer post hoc analysis) (FIG. 18A). Treatment with either inhibitor or their combination resulted in significantly lower MMP-9 expression (FIG. 18C) (p<0.0001, ANOVA Tukey Kramer post hoc analysis). MK-801 alone also caused a significant reduction in MMP-2 expression compared to PA+NE-tPA treatment (FIG. 2B) (p<0.05, ANOVA Tukey Kramer post hoc analysis). MMP-13 expression values were similar in all groups (FIG. 18D) (p>0.05, ANOVA Tukey-Kramer post hoc analysis).

Simulated Outflow facility is further illustrated in FIG. 18A in ACOS with HTM treated with PA+NE-tPA, PA+NE-tPA+MK-801, PA+NE-tPA+RAP and PA+NE-tPA+MK-801+RAP (****p<0.0001, ANOVA, Tukey-Kramer post hoc analysis). Gene expression of MMP-2 (FIG. 18B), MMP-9 (FIG. 18C), and MMP-13 (FIG. 18D) are normalized (mean±SD) to values in PA+NE-tPA treated cells. MMP-2 expression was significantly lower in PA+NE-tPA+MK-801 treated cells compared to PA+NE-tPA treated cells. MMP-9 expression was significantly lower in PA+NE-tPA+MK-801, PA+NE+RAP, and PA+NE-tPA+MK-801+RAP treated cells compared to PA+NE-tPA treated cells (ANOVA MMP-2, p<0.05, MMP-9, p<0.0001, MMP-13, p>0.05). *p<0.05, **p<0.01, ***p<0.001. ****p<0.0001.

### Example 19-Whether Tissue Plasminogen Activator Attenuates Outflow Facility Reduction in Mouse Model of Juvenile Open Angle Glaucoma

### 19A. Methods:

### 19A.1-Animals and Treatments

8- to 12-week-old male and female mice were used for this study. The animals were housed and bred at the State University of New York (SUNY) Downstate Health Sciences University Division of Comparative Medicine (Brooklyn, NY) under a 12-hour light/12-hour dark cycle and were fed ad libitum. A Tg-MYOCY4371-1 mouse colony was established from animals provided by Dr. Gulab Zode. These mice contain the transgenic human MYOC gene, with a Tyr437His mutation. Protocols were approved by the SUNY Downstate Institutional Animal Care and Use Committee, and experiments were performed according to the ARVO Statement for the Use of Animals in Ophthalmic and Vision Research.

To investigate whether corticosteroids affect outflow facility in Tg-MYOCY437H mice, animals received bilateral injections (20 μl) with either triamcinolone acetonide (TA) (40 mg/ml, Kenalog-40; Bristol-Myers Squibb, NY, USA) suspension or phosphate-buffered saline (PBS) (Gibco, ThermoFisher Scientific, Waltham, MA, USA) subconjunctivally using a 100 μL Hamilton syringe with a 26-gauge needle (Precision Glide, Becton Dickinson & CO, Franklin Lakes, NJ, USA). They were euthanized one week later for outflow facility measurement.

To investigate whether tPA can affect outflow facility in Tg-MYOCY437H mice, and determine whether tPA enzymatic action is necessary for such an effect, intravitreal injections were performed using a 10 μL Hamilton syringe with 36-gauge stainless steel needles (WPI Inc, Sarasota, FL, USA). Animals received unilateral intravitreal injections (2 μl, 5 μg/μl) of either tissue plasminogen activator (tPA)(Actilyse; Boehringer Ingelheim, Ingelheim am Rhein, Germany) or enzymatically inactive tissue plasminogen activator (NE-tPA/S478A-tPA) (Innovative Research, Novi, MI, USA) while the contralateral eye received bovine serum albumin (BSA) (2 μl of 5 μg/μl; Gold Biotechnology, St Louis, MO, USA). Animals were sacrificed either two or five days after intravitreal injections for outflow facility measurement. All injections were performed under isoflurane inhalation anesthesia and topical anesthesia with 0.5% proparacaine.

To compare the efficacy of tPA to that of sodium phenylbutarate (PBA) (MilliporeSigma, Burlington, MA, USA), PBA was dissolved in sterile PBS to make a 0.2% solution. Fresh solution was made weekly and stored at room temperature. Tg-MYOCY437H mice received bilateral topical ocular PBA twice daily for five days prior to euthanasia. A single drop of 50 μl PBA solution was instilled into each eye under isoflurane inhalation anesthesia for each drop administration.

### 19A.2-IOP Measurement

IOP was measured with a rebound tonometer after application of 0.5% proparacaine topical anesthesia while animals were restrained in a custom-made device (Danias, Kontiola et al. 2003, Kumar, Shah et al. 2013). Five measurements were averaged per each eye. Measurements were made between 10 AM and 12 PM, to minimize the effect of diurnal IOP variation.

### 19A.3-Outflow Facility Determination

Mouse eyes were enucleated immediately after euthanasia. Outflow facility was determined using a microfluidic flow sensor (0.07-1.5 uL/min, MFS1; Elveflow, Paris, France) and constant pressure method, as previously described (Hu, Barron et al. 2019). Flow rates were plotted at unique pressure levels and the slope of the regression line was used to calculate the outflow facility for each eye. Any eyes that developed visible leaks or that had pressure-flow correlations with R2 0.9 were excluded from analysis but were used for RNA quantification.

### 19A.4-Tissue Collection, RNA Isolation and Quantitative Real Time PCR

Eyes were flash frozen in liquid nitrogen after outflow facility determination and subsequently dissected on ice to obtain the angle ring tissues containing the TM, as previously described (Kumar, Shah et al. 2013). Dissected TM tissues were immersed in RNAlater solution (Invitrogen, Waltham, MA, USA) and then snap-frozen and stored at −80° C. until RNA extraction. Tissue collected was pooled (four eyes) and homogenized in TRIzol reagent for RNA isolation, per manufacturer's instructions (Life Technologies, Carlsbad, CA, USA). RNA concentration was determined with a Nanodrop ND-1000 Spectrophotometer (Thermo Scientific, Wilmington, DE, USA). cDNA was synthesized by using High-Capacity cDNA

Reverse Transcription Kit (Applied Biosystems, Waltham, MA, USA) according to manufacturer's protocol. Quantitative real-time PCR (qRT-PCR) was performed by using Green-2-Go qPCR Mastermix-ROX (Bio Basic, Amherst, NY, USA) on a QuantStudio 6 Flex thermal cycler (Applied Biosystems, Carlsbad, CA, USA). mRNA expression of Plat, Plait, Pai-1, Mmp-2, Mmp-9, and Mmp-13 in angle ring tissues was determined. Primers were designed by using Primer-BLAST (haps://www.ncbi.nlm.nih.gov/tools/primer-blast/index.cgi?LINK_LOC=BlastHome; in the public domain); primer sequences are listed in Table 2. Primer specificity was confirmed by agarose gel electrophoresis and multiple-sized amplification products were absent on inspection of melting curves. Target mRNA expression values were normalized to the expression levels of Rps11 (ribosomal protein S11). The relative fold change was calculated by using the ΔΔCt method (Hu, Wang et al. 2015). Outliers identified via the Thompson Tau test were removed from analysis.

TABLE 2

| Number | Gene | Sequence (5′-3′) |
|---|---|---|
| 1 | Plat | FP: CAGTGCCCAGAAGGGTTCAT |
| | | RP: GTAGCACCAGGGCTTTGAGT |
| 2 | Plau | FP: GCGCCTTGGTGGTGAAAAAC |
| | | RP: GACACGCATACACCTCCGTT |
| 3 | Pai-1 | FP: ATGATGGCTCAGAGCAACAAG |
| | | RP: CATTGTCTGATGAGTTCAGCATC |
| 4 | Mmp-2 | FP: ACAGTGACACCACGTGACAA |
| | | RP: GGTCAGTGGCTTGGGGTATC |
| 5 | Mmp-9 | FP: GCGTCGTGATCCCCACTTAC |
| | | RP: CAGGCCGAATAGGAGCGTC |
| 6 | Mmp-13 | FP: TACCATCCTGCGACTCTTGC |
| | | RP: TTCACCCACATCAGGCACTC |
| 7 | Rps11 | FP: AAGACGCCTAAAGAGGCTATTG |
| | | RP: GGTCCTCTGCATCTTCATCTTC |

FP, forward prime; RP, reverse primer

### 19B. Results:

### 19B.1-Outflow Facility and Expression of Fibrinolytic Pathway Components in Tg-MYOC Y437H Mice

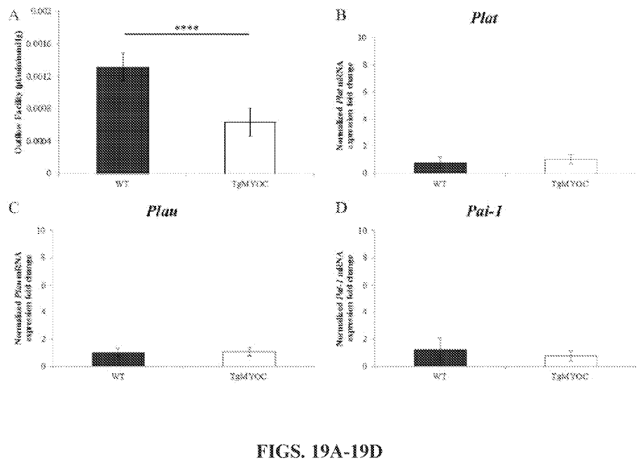

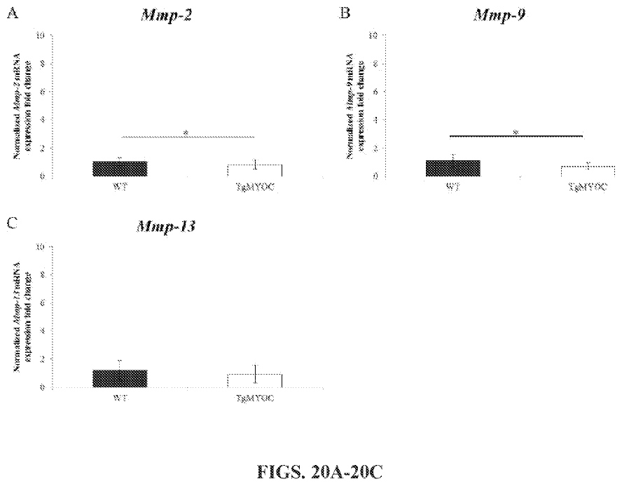

The mean±standard deviation outflow facility (μl/min/mmHg) was 63±17×10-5 in Tg-MYOCY437H (n=10) mice and 132±17.2×10-5 in wildtype (WT) littermate mice (n=6). The Tg-MYOCY437H mouse eyes had a significantly (˜52%) lower outflow facility compared to WT littermate eyes (p<0.0001, T-test) (FIG. 19A). IOP was also significantly (˜47%) elevated in Tg-MYOCY437H mouse eyes compared to WT eyes (p<0.05, T-test; data not shown). There were no significant differences in expression of Plat (FIG. 19B), Plau (FIG. 19C) and Pai-1 (FIG. 19D) in angle ring tissue of eyes fromTg-MYOCY437H (n=11) and WT littermate (n=9) mice (p>0.05, Ttest). However, Tg-MYOCY437H mice (n=11) had significantly lower angle ring tissue expression of Mmp-2 (FIG. 20A) and Mmp-9 (FIG. 20B) compared to that of WT (n=9) littermates (p<0.05 and p<0.05. T-test respectively). There was no significant difference in Mmp-13 (FIG. 20C) expression (p>0.05, T-test) between Tg-MYOCY437H mice and WT littermates. 19B.2-Effect of Steroids on Tg-MYOC Y437H Mouse Outflow Facility

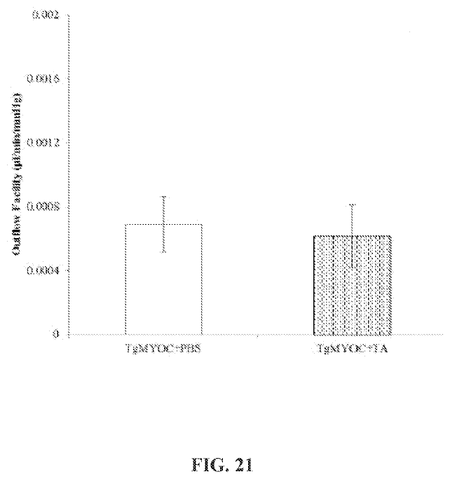

Outflow facility (mean±standard deviation) in Tg-MYOCY437Hmouse eyes treated with TA (n=7) was 61±20×10-5 μl/min/mmHg while that in Tg-MYOCY437H mouse eyes treated with PBS (n=10) was 69±17×10-5 μl/min/mmHg. Outflow facility was not significantly different between TA and PBS treated groups (p>0.05, T-test) (FIG. 21).

### 19B.3-Effect of Enzymatically Active and Enzymatically Inactive Tissue Plasminogen Activator on Outflow Facility in Tg-MYOCY437H Mice

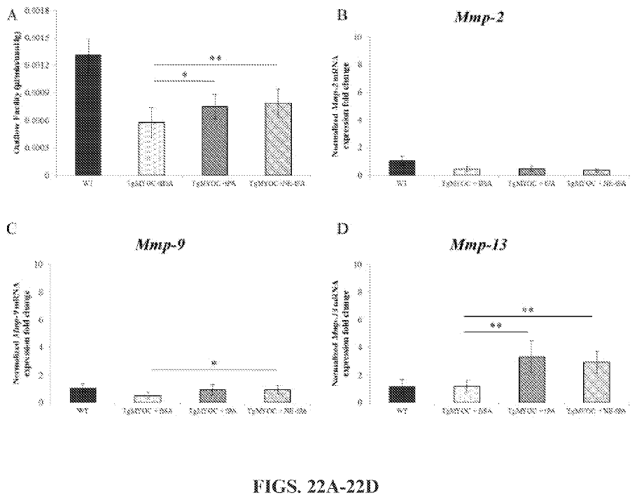

In Tg-MYOCY437H eyes 2 days after treatment with the respective protein injection, the mean±standard deviation outflow facility (μl/min/mmHg) was 57.4±16.8×10-5 in BSA treated (n=17), 75±13.7×10-5 in tPA-treated (n=10) and 78.6±15.6×10-5 in NE-tPA-treated (n=8) groups (p<0.0001, ANOVA). Treatment with tPA or NE-tPA significantly enhanced outflow facility compared to BSA treatment (p<0.05 and p<0.01, respectively, Tukey-Kramer post hoc analysis), but did not improve outflow facility to that of WT littermate mouse eyes (132±17.2) (n=6). There was no significant difference between tPA and NE-tPA treatments (p>0.05. Tukey-Kramer post hoc analysis) (FIG. 22A).

Significant differences in the expression of Mmp-2, Mmp-9 and Mmp-13 between WT BSA, tPA and NE-tPA groups were detected (ANOVA, p<0.0001, p<0.01, and p<0.0001, respectively). NE-tPA treated Tg-MYOCY437H eyes (n=8) showed significantly higher Mmp-9 expression compared to BSA treated eyes (n=16) (p<0.05, Tukey-Kramer post hoc analysis)(FIG. 22C). tPA (n=8) and NE-tPA (n=8) treated Tg-MYOCY437H eyes showed significantly higher Mmp-13 expression compared to BSA treated eyes (n=16) (p<0.01 and p<0.01, respectively, Tukey-Kramer post hoc analysis)(FIG. 22D). There was no significant difference in Mmp-2 (FIG. 22B) expression when comparing NE-tPA (n=8) and tPA (n=8) treated eyes to BSA (n=16) treated eyes (p>0.05, Tukey-Kramer post hoc analysis). Mmp-2 expression remained decreased in BSA, tPA and NE-tPA groups compared to that in WT littermate mice (p<0.0001, p<0.01, p<0.0001, respectively, Tukey-Kramer post hoc analysis).

### 19B.4-Comparison of the Effectiveness of Tissue Plasminogen Activator and Sodium Phenylbutarate in Improving Outflow Facility in Tg-MYOCY437H Mice

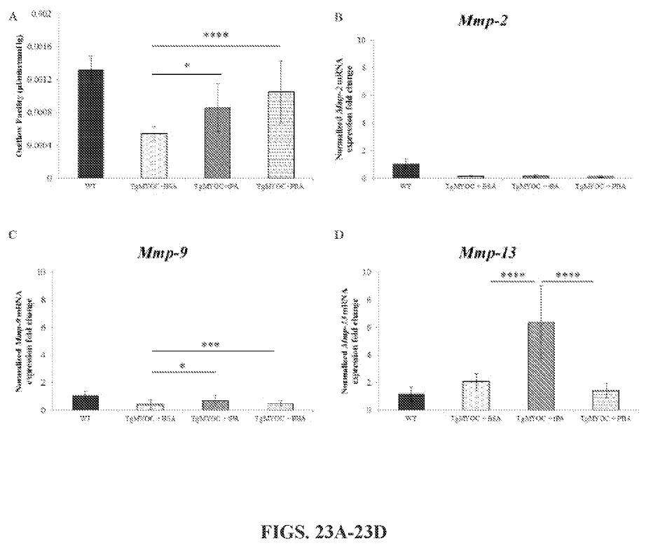

In Tg-MYOCY437H eyes 5 days after treatment with intravitreal protein, the mean±standard deviation outflow facility (μl/min/mmHg) was 85.4±28.8×10-5 in tPA-treated (n=13) and 54±8.3×10-5 in BSA-treated (n=15) eyes. In Tg-MYOCY437H eyes treated with topical 0.2% PBA, the mean±standard deviation outflow facility was 105±37.8×10-5 (n=14) (p<0.0001, ANOVA). Treatment with either intravitreal tPA or topical PBA significantly enhanced outflow facility compared to BSA treated eyes (p<0.05 and p<0.0001, respectively; Tukey-Kramer post hoc analysis). There was no significant difference between tPA and PBA treated eyes (p>0.05, Tukey-Kramer post hoc analysis) (FIG. 23A).

Expression of Mmp-2, Mmp-9 and Mmp-13 was significantly different between WT, BSA, tPA and PBA groups (ANOVA, p<0.0001, p<0.001 and p<0.0001, respectively). Although, Mmp-2 expression was lower in BSA, tPA and PBA groups compared to WT littermates (p<0.0001, p<0.0001, p<0.0001, respectively, Tukey-Kramer post hoc analysis) there was no significant difference in Mmp-2 expression when comparing tPA (n=8) and PBA (n=8) treated eyes to BSA (n=8) treated eyes (p>0.05, Tukey-Kramer post hoc analysis)(FIG. 23B). Both tPA (n=8) and PBA (n=8) treated eyes had higher Mmp-9 expression (FIG. 23C) compared to BSA treated eyes (n=8) (p<0.05 and p<0.001, respectively, Tukey-Kramer post hoc analysis). tPA-treated Tg-MYOCY437H eyes (n=8) showed a significantly higher Mmp-13 expression compared to BSA treated eyes (n=8) and PBA treated eyes (n=8) (p<0.0001 and p<0.0001, respectively, Tukey-Kramer post hoc analysis) (FIG. 23D). PBA treated eyes (n=8) did not show any difference in Mmp-13 expression from that of BSA-treated and WT eyes (p>0.05, Tukey-Kramer post hoc analysis) (FIG. 23D).

The described embodiments and examples of the present disclosure are intended to be illustrative rather than restrictive, and are not intended to represent every embodiment or example of the present disclosure. While the fundamental novel features of the disclosure as applied to various specific embodiments thereof have been shown, described and pointed out, it will also be understood that various omissions, substitutions and changes in the form and details of the devices illustrated and in their operation, may be made by those skilled in the art without departing from the spirit of the disclosure. For example, it is expressly intended that all combinations of those elements and/or method steps which perform substantially the same function in substantially the same way to achieve the same results are within the scope of the disclosure. Moreover, it should be recognized that structures and/or elements and/or method steps shown and/or described in connection with any disclosed form or embodiment of the disclosure may be incorporated in any other disclosed or described or suggested form or embodiment as a general matter of design choice. Further, various modifications and variations can be made without departing from the spirit or scope of the disclosure as set forth in the following claims both literally and in equivalents recognized in law.

What is claimed is:

1. A method of increasing outflow facility in a subject, the method comprising:
administering to the subject an effective amount an enzymatically inactive tPA variant therapeutic agent.

2. The method of claim 1, wherein the enzymatically inactive tPA variant therapeutic agent is selected from the group consisting of an enzymatically inactive tPA protein and a vector encoding an enzymatically inactive tPA variant.

3. The method of claim 1, wherein administering the enzymatically inactive tPA variant therapeutic agent reduces intra ocular pressure (IOP) in said subject for a period of at least one day to a year or more, relative to IOP measurements in said subject prior to administration of the enzymatically inactive tPA variant therapeutic agent.

4. The method of claim 3, wherein the administration comprises administering the enzymatically inactive tPA variant therapeutic agent to said subject for a period of at least two weeks.

5. The method of claim 1, wherein the subject has open angle glaucoma.

6. The method of claim 1, wherein the subject has human juvenile open angle glaucoma.

7. The method of claim 1, wherein the enzymatically inactive tPA variant therapeutic agent is administered by a method selected from the group consisting of intracameral injection, intravitreal injection, topical application, systemic administration, an implantable device, and an implantation of cells that produce said enzymatically inactive tPA variant therapeutic agent.

8. A method of treatment for chronic elevated intraocular pressure (IOP) in a subject, the method comprising:
administering to the subject an effective amount of an enzymatically inactive tPA variant therapeutic agent.

9. The method of claim 8, wherein the enzymatically inactive tPA variant therapeutic agent is selected from the group consisting of an enzymatically inactive tPA protein and a vector encoding an enzymatically inactive tPA variant.

10. The method of claim 8, wherein administering the enzymatically inactive tPA variant therapeutic agent reduces intra ocular pressure (IOP) in said subject for a period of at least one day to a year or more, relative to IOP measurements in said subject prior to administration of the enzymatically inactive tPA variant therapeutic agent.

11. The method of claim 10, wherein the administration comprises administering the enzymatically inactive tPA variant therapeutic agent to said subject for a period of at least two weeks.

12. The method of claim 8, wherein the subject has open angle glaucoma.

13. The method of claim 8, wherein the subject has human juvenile open angle glaucoma.

14. The method of claim 8, wherein the enzymatically inactive tPA variant therapeutic agent is administered by a method selected from the group consisting of intracameral injection, intravitreal injection, topical application, systemic administration, an implantable device, and an implantation of cells that produce said enzymatically inactive tPA variant therapeutic agent.
